(19) United States
(12) Patent Application Publication
(10) Pub. No.: US 2025/0000820 A1
(43) Pub. Date: Jan. 2, 2025

(54) ORAL TABLET WITH IMPROVED UNIFORMITY OF CONTENT OF ACTIVES

(71) Applicant: FERTIN PHARMA A/S, Vejle (DK)

(72) Inventors: Bruno Provstgaard Nielsen, Vejle O (DK); Helle Martinussen, Aarhus C (DK)

(21) Appl. No.: 18/343,849

(22) Filed: Jun. 29, 2023

(51) Int. Cl. A61K 31/137; A61K 9/00; A61K 9/20; A61K 31/167; A61K 31/485

(52) U.S. Cl. CPC A61K 31/137; A61K 9/0056; A61K 9/2018; A61K 9/2054; A61K 9/2059; A61K 9/2086; A61K 31/167; A61K 31/485

(57) ABSTRACT

The invention relates an oral tablet suitable for active pharmaceutical ingredients comprising an intragranular portion and an extragranular portion. The intragranular portion comprising a plurality of granules each comprising one or more active pharmaceutical ingredients; one or more binders; and one or more excipients comprising one or more sweeteners in an amount of at least 20% by weight of the plurality of granules. The extragranular portion comprising a population of particles comprising non-directly compressible (non-DC) sweetener particles, the non-DC sweetener particles having not been granulated prior to tableting and providing the oral tablet with a plurality of discrete non-DC areas evenly distributed in the oral tablet or at least one module of the oral tablet.

### TECHNICAL FIELD

The present invention relates to the field of oral tablets comprising distant portions of tablet material suitable for delivery of active pharmaceutical ingredients. In particular, the invention relates to tablets having an intragranular portion serving to improve content uniformity of one or more active pharmaceutical ingredients together with an extragranular portion serving to improve saliva generation.

### BACKGROUND

Various attempts have been made in the past with an aim to improve content uniformity of active pharmaceutical ingredients in tablets in order to secure accurate dosing for patients in need thereof. Accurate dosing may be important for certain actives in order to avoid overdosing with potentially lethal consequences and also to provide precise vehicles for administration of actives. Uniformity of content is of particular importance for active pharmaceutical ingredients where lack of safety and appropriate delivery may become fatal in alleviating or treating medical conditions.

Mainly relevant for tablets intended for delivery of actives in the gastrointestinal tract, focus has traditionally been on formulating encapsulations for targeted delivery of the actives in the gastrointestinal tract. Suitable solutions for administration of active pharmaceutical ingredients with high efficacy in tablets for delivery in the oral cavity has been particularly challenging since such actives are usually to be provided in low doses.

In parallel, oral tablets have been provided in the past with special focus on providing increased saliva generation and thereby promoting release of active ingredients from the oral tablets to the oral cavity. These tablets have mainly been intended for delivery and uptake of actives through the oral mucosa upon release of the actives in the oral cavity. While improved saliva generation may be obtained by these tablets and fast release of the actives may be accommodated, certain challenges may arise with respect to accurate dosing of certain actives due to increased saliva production. Uncontrolled delivery of actives may inherently be increased as a consequence of too much deviation from the targeted content of the actives in such tablets, e.g., a less beneficial content uniformity of the actives.

Combining means for an improved content uniformity of actives, such as low dose actives, with increased saliva generation may be particularly desired but has hitherto been difficult to obtain due to the intrinsic opposite function of these systems.

Traditionally, means for improved content uniformity, such as encapsulations, may not be compatible with a desire to have increased saliva generation and thereby improved delivery of actives in the oral cavity. This may for example be the case where the means for an improved content uniformity prevents delivery to the oral mucosa upon increased saliva generation. Also, this may be the case where the means for obtaining an improved content uniformity prevents increased saliva generation, even if means as present for increased saliva generation.

Particularly, it may be a challenge to formulate saliva promoting oral tablets with active pharmaceutical ingredients in low doses which may be required for actives where the efficacy is high and would otherwise cause problems if provided in a higher dose. In that case, the oral tablet means for improved content uniformity of the actives may be compromised by the formulation of the saliva promoting means in the oral tablets. This may be the case, where the means for saliva promotion is based on a combination of non-directly compressible (non-DC) and directly compressible (DC) sweetener particles and the means for improved content uniformity is based on means that may counteract saliva promotion.

Oral tablets have traditionally been preferred for administration of active pharmaceutical ingredients both in terms of convenience and compliance. Oral tablets have certain benefits compared to other delivery vehicles for oral administration of active ingredients. Typically, such oral tablets are made by direct compression or compaction methods where a powder tablet material and an active ingredient are pressed into defined tablets with appropriate strength to provide a pharmacological effect to a patient in need thereof in medical formulations or to provide a health benefit for consumers in nutraceutical formulations.

In terms of low dose active pharmaceutical ingredients, and even high dose active pharmaceutical ingredients, one option is to apply such actives in an oral tablet made by direct compression without special means for providing a high content uniformity of the actives. However, due to the process of tablet manufacturing, a challenge of powder segregation may apply. This may result in a less beneficial content uniformity in the oral tablet made by the process. And this may also cause issues in terms of the overall formulation of the oral tablets, such as problems to the saliva promoting means since the various components of the oral formulation are intrinsically linked together.

In spite of the efforts and previous improvements of formulating oral tablets, hitherto known oral tablets are associated with various drawbacks. For instance, the time delay from administration of oral tablets to full efficacy of the active ingredient may inherently be delayed since the active ingredients are usually released over time from the oral tablet. This applies for instance when the tablet is designed for buccal absorption or gastrointestinal delivery.

Additionally, convenience may be considerably compromised for certain consumers by formulating active ingredients in oral tablets. Here, aspects such as problems with swallowing tablets become critical, for instance for people with dry mouth and reduced saliva generation. Eventually, this may result in poor treatment for medical patients or poor health benefits for consumers of nutraceutical ingredients.

Particularly, only minor attention is given to benefits that may help obtaining release characteristics of active ingredients resulting in increased convenience and effectiveness. One of these release characteristics is increased generation of saliva. Increased generation of saliva and particularly an experience of increased saliva generation upon administration may for instance have some pronounced benefits for delivery of active ingredients to mucosal surfaces.

Furthermore, it is preferable that a formulation is provided that may also help in obtaining improved sensorial properties of active ingredient delivery. Here, important sensorial properties include mouthfeel, melting sensation, flavor sensation, salivation, cooling sensation, and off-note sensation associated with active ingredients. These properties are both relevant from a convenience perspective in oral administration, but certainly also in order to support an appropriate delivery of active ingredients and avoid adverse side effects of active ingredients. In particular mouthfeel is one of the more important sensorial properties of active ingredient delivery apart from efficacy.

One of the challenges with oral tablets as a delivery vehicle of active ingredients is that some active ingredients tend to be associated with off-notes during administration due to specific physiochemical properties. Taste masking challenges are more profound when a higher release of such active ingredients is required. If off-notes are the predominant sensation during administration, convenience may be affected and even more critically, delivery of such active ingredients may also be affected.

Hence, there is a need in the prior art for improved administration platforms that solve the above-referenced challenges and problems of the prior art. In particular, there is a need in the art for new platforms that support improved saliva generation combined with appropriate content uniformity of active pharmaceutical ingredients and beneficial sensorial properties.

### SUMMARY

The present invention pertains to an oral tablet suitable for active pharmaceutical ingredients comprising an intragranular portion and an extragranular portion. The intragranular portion comprises a plurality of granules each comprising: one or more active pharmaceutical ingredients; one or more binders; and one or more excipients comprising one or more sweeteners in an amount of at least 20% by weight of the plurality of granules. The extragranular portion comprises a population of particles comprising: non-directly compressible (non-DC) sweetener particles, the non-DC sweetener particles having not been granulated prior to tableting and providing the oral tablet with a plurality of discrete non-DC areas evenly distributed in the oral tablet or at least one module of the oral tablet.

One advantage of the present invention is a surprisingly strong saliva generation compared to conventional chewable tablets and lozenges together with an advantageous content of uniformity of the active pharmaceutical ingredients.

In terms of saliva generation, the plurality of non-DC areas provided in the tablet surprisingly induces a remarkable generation of saliva and provides a synergy in release the active pharmaceutical ingredients of the intragranular portion of the oral tablet. Having a combination of an extragranular portion and an intragranular portion according to the invention may further facilitate sufficient mechanical strength combined with stability of the tablet, disintegrability of the tablet upon chewing, and induced saliva generation upon chewing.

One further advantage in terms of saliva generation is that the saliva generation is surprisingly sustained even after a user has swallowed the bulk-portion of the non-DC sweeteners. This sustaining of the salivation generation may be advantageous in relation to many applications of an oral tablet ranging from mouthfeel, taste, flavor perception, etc. Additionally, it may work in synergy with the active pharmaceutical ingredients of the intragranular portion.

Combining the intragranular portion with the extragranular portion according to the invention provides at the same time increased saliva production and improved content uniformity of the actives. This combination is particularly advantageous in a formulation with low dose active pharmaceutical ingredients but may also be advantageous for high dose active pharmaceutical ingredients.

In terms of improved content of uniformity, it was surprisingly seen that the intragranular portion and the extragranular portion work in synergy according to the invention. While the intragranular portion according to the invention serves to provide a suitable content of uniformity, the extragranular portion according to the invention serves to provide saliva generation without compromising the delivery of the actives. To the contrary, the system promotes delivery of the actives, even when formulated with high dose actives. Beneficial saliva production is promoted and sufficient content of uniformity is provided according to the invention.

Special mechanical advantages are provided by combining the intragranular portion with the non-directly compressible (non-DC) sweetener particles according to the invention. While the non-DC sweetener particles promotes saliva generation, the intragranular portion provides sufficient compressibility to the oral tablet. This was a surprise to the inventors, since it was expected that the non-DC sweetener particles would contribute to a brittle tablet that could not be sufficiently compressed even in the presence of an intragranular portion according to the invention.

Generally, the oral tablet of the present invention unlike traditional oral tablets may be associated with various benefits in terms of sensorial properties and various other properties, such as release properties. The oral tablet is designed to encompass a synergistic combination of different types of sweetener particles, such as sugar alcohol particles. Combined, the different types of sweetener particles serve to both deliver active ingredients with improved effect and to accommodate various sensorial benefits compared to conventional oral tablets, including improved mouthfeel. Also, the oral tablet of the present invention is aimed to be superior compared to simpler and less intricate oral tablets available for administration of active ingredients.

Particularly, the present invention may help in obtaining a release characteristic of active ingredients that offers increased convenience and effectiveness. One of these release characteristics is increased generation of saliva. Increased saliva generation and particularly an experience of increased saliva generation upon administration may for instance have some pronounced benefits for delivery of active ingredients to mucosal surfaces.

Furthermore, the present invention may help in obtaining improved sensorial properties of active ingredient delivery. Here, important sensorial properties include mouthfeel, melting sensation, flavor sensation, salivation, cooling sensation, and off-note sensation associated with active ingredients or processing aids. Of particular concern is to provide a suitable mouthfeel in order to allow medical patients or consumers seeking health benefits a more accommodating treatment or alleviation of symptoms. Also, the present invention may help in improving taste-masking of off-notes during administration. The taste masking challenge is more profound when a higher release of such active ingredients are provided which is generally the case for the system of the present invention.

In some embodiments of the invention, the oral tablet is a chewable tablet.

In context of the present invention, a “chewable tablet” is intended to mean an oral tablet that is chewed upon oral administration, having characteristics allowing convenient chewing without adverse side effects associated with the texture of the oral tablet.

In some embodiments of the invention, the extragranular portion constitutes at least 20% by weight of the oral tablet. In some embodiments of the invention, the extragranular portion constitutes at least 30% by weight of the oral tablet. In some embodiments of the invention, the extragranular portion constitutes at least 40% by weight of the oral tablet. In some embodiments of the invention, the extragranular portion constitutes at least 50% by weight of the oral tablet. In some embodiments of the invention, the extragranular portion constitutes at least 60% by weight of the oral tablet.

In some embodiments of the invention, the extragranular portion constitutes 20-60% by weight of the oral tablet. In some embodiments of the invention, the extragranular portion constitutes 20-50% by weight of the oral tablet. In some embodiments of the invention, the extragranular portion constitutes 20-40% by weight of the oral tablet. In some embodiments of the invention, the extragranular portion constitutes 30-60% by weight of the oral tablet. In some embodiments of the invention, the extragranular portion constitutes 30-50% by weight of the oral tablet. In some embodiments of the invention, the extragranular portion constitutes 40-60% by weight of the oral tablet. In some embodiments of the invention, the extragranular portion constitutes 40-70% by weight of the oral tablet. In some embodiments of the invention, the extragranular portion constitutes 40-80% by weight of the oral tablet. In some embodiments of the invention, the extragranular portion constitutes 50-80% by weight of the oral tablet. In some embodiments of the invention, the extragranular portion constitutes 60-80% by weight of the oral tablet.

In some embodiments of the invention, the intragranular portion constitutes at least 30% by weight of the oral tablet. In some embodiments of the invention, the intragranular portion constitutes at least 40% by weight of the oral tablet. In some embodiments of the invention, the intragranular portion constitutes at least 50% by weight of the oral tablet. In some embodiments of the invention, the intragranular portion constitutes at least 60% by weight of the oral tablet. In some embodiments of the invention, the intragranular portion constitutes at least 70% by weight of the oral tablet.

In some embodiments of the invention, the intragranular portion constitutes 30-70% by weight of the oral tablet. In some embodiments of the invention, the intragranular portion constitutes 30-60% by weight of the oral tablet. In some embodiments of the invention, the intragranular portion constitutes 30-50% by weight of the oral tablet. In some embodiments of the invention, the intragranular portion constitutes 40-70% by weight of the oral tablet. In some embodiments of the invention, the intragranular portion constitutes 40-60% by weight of the oral tablet. In some embodiments of the invention, the intragranular portion constitutes 50-70% by weight of the oral tablet. In some embodiments of the invention, the intragranular portion constitutes 50-80% by weight of the oral tablet. In some embodiments of the invention, the intragranular portion constitutes 60-80% by weight of the oral tablet.

In some embodiments of the invention, the plurality of discrete non-DC areas of the extragranular portion provide increased saliva generation upon mastication of the oral tablet.

In the present context when the non-DC areas are referred to as “discrete” this signifies that the non-DC sweeteners are not continuously distributed, but present in the discrete areas corresponding to the discrete nature of the non-DC sweetener particles.

In the present context, the term “non-DC areas” refers to small volumes or spaces formed during tableting from the non-DC particles of non-DC sugar alcohol. Moreover, each of the non-DC areas may be composed of a single non-DC sugar alcohol particle, or may comprise several non-DC sugar alcohol particles. When the non-DC areas are distinct, i.e. not diffuse, the non-DC areas may be evenly distributed in the tablet, or at least one module thereof when the tablet comprises two or more modules. In such embodiments, where the non-DC areas are evenly distributed in in the tablet, or at least one module thereof, the non-DC areas may thus facilitate an even saliva generation in the mouth upon mastication.

When referring to increased saliva generation, it is noted that this increased saliva generation exceeds any saliva generation without the use of the tablet of the invention. Particularly, in an embodiment the induced saliva generation exceeds saliva generation when using conventional tablets without non-DC areas. Then, induced saliva generation is increased over any saliva generation associated with conventional products, e.g. by comparing with a tablet without non-DC sweetener particles, or with a tablet where the discrete areas are based on DC sweetener particles.

When referring to increased saliva generation, the saliva generation is tested using the following method, unless stated otherwise.

Test subject abstain from eating and drinking at least 30 minutes before initiation of any test. Immediately before introducing of the tablet into the oral cavity, the test subject swallows. The test subject refrains from swallowing during the test. Immediately after introducing of the tablet into the oral cavity, the test subject starts masticating the tablet at a frequency of 1 chew per second for 20 seconds. Then, saliva and any remains of the tablet is kept in the mouth within chewing for 10 second. 30 seconds after starting the test, the test subject discards saliva including any tablet fragments into a plastic cup, which is weighted. Saliva discarded also at 90 seconds after onset of mastication, at 180 seconds after onset of mastication, at 300 seconds after onset of mastication, at 420 seconds after onset of mastication, and at 600 seconds after onset of mastication. At all times, the test subject makes as little movement as possible, and refrains from swallowing.

It is noted that the non-DC particles may form small sub-areas or sub spaces in the final oral tablet or the relevant module of the final tablet. These sub-areas may be referred to as discrete non-DC areas and may be formed by single non-DC particles or very small groups of these non-DC particles.

In an embodiment of the invention, the tablet generates more than 1.5 mL saliva within 30 seconds from onset of mastication. According to an embodiment of the invention the discrete non-DC areas induces saliva generation of more than 2.0 mL saliva within 30 seconds from onset of mastication. According to an embodiment of the invention the discrete non-DC areas induces saliva generation of more than 3.0 mL saliva within 30 seconds from onset of mastication. In an embodiment of the invention, the tablet generates more than 1.5 mL saliva within a period from 30 to 90 seconds from onset of mastication. According to an embodiment of the invention the discrete non-DC areas induces saliva generation of more than 2.0 mL saliva within 30 to 90 seconds from onset of mastication. In an embodiment of the invention, the tablet generates more than 1.5 mL saliva within a period from 90 to 180 seconds from onset of mastication. In an embodiment of the invention, the tablet generates more than 1.5 mL saliva within a period from 180 to 300 seconds from onset of mastication.

In some embodiments of the invention, saliva generation upon mastication of the oral tablet is increased compared to an oral tablet where the discrete areas of the extragranular portion are based on DC sweetener particles.

In some embodiments of the invention, saliva generation upon mastication of the oral tablet is increased compared to an oral tablet where the discrete areas of the extragranular portion is free of non-DC sweetener particles.

In some embodiments of the invention, the non-DC sweetener particles of the extragranular portion is present in an amount of at least 10% by weight of the oral tablet. In some embodiments of the invention, the non-DC sweetener particles of the extragranular portion is present in an amount of at least 20% by weight of the oral tablet. In some embodiments of the invention, the non-DC sweetener particles of the extragranular portion is present in an amount of at least 30% by weight of the extragranular portion. In some embodiments of the invention, the non-DC sweetener particles of the extragranular portion is present in an amount of at least 40% by weight of the extragranular portion.

In some embodiments of the invention, the non-DC sweetener particles of the extragranular portion is present in an amount of 10-50% by weight of the oral tablet. In some embodiments of the invention, the non-DC sweetener particles of the extragranular portion is present in an amount of 10-40% by weight of the oral tablet. In some embodiments of the invention, the non-DC sweetener particles of the extragranular portion is present in an amount of 10-30% by weight of the oral tablet. In some embodiments of the invention, the non-DC sweetener particles of the extragranular portion is present in an amount of 20-50% by weight of the oral tablet. In some embodiments of the invention, the non-DC sweetener particles of the extragranular portion is present in an amount of 30-50% by weight of the oral tablet. In some embodiments of the invention, the non-DC sweetener particles of the extragranular portion is present in an amount of 30-50% by weight of the oral tablet.

In some embodiments of the invention, the non-DC sweetener particles of the extragranular portion comprise one or more non-DC sugar alcohol particles.

Preferably, the non-DC sweetener particles of the extragranular portion comprise one or more non-DC sugar alcohol particles.

In some embodiments of the invention, the non-DC sweetener particles of the extragranular portion are selected from the group consisting of non-DC particles of erythritol, maltitol, xylitol, isomalt, lactitol, mannitol, and combinations thereof.

In some embodiments of the invention, the non-DC sweetener particles of the extragranular portion are selected from the group consisting of non-DC particles of erythritol, maltitol, xylitol, isomalt, mannitol, and combinations thereof.

In some embodiments of the invention, the non-DC sweetener particles of the extragranular portion are selected from the group consisting of non-DC particles of erythritol, maltitol, xylitol, isomalt, lactitol, and combinations thereof.

In some embodiments of the invention, the non-DC sweetener particles of the extragranular portion are selected from the group consisting of non-DC particles of erythritol, maltitol, xylitol, isomalt, and combinations thereof.

In some embodiments of the invention, the non-DC sweetener particles of the extragranular portion are selected from the group consisting of non-DC particles of erythritol, maltitol, xylitol, and combinations thereof.

In some embodiments of the invention, the non-DC sweetener particles comprise erythritol. In some embodiments of the invention, the non-DC sweetener particles comprise maltitol. In some embodiments of the invention, the non-DC sweetener particles comprise xylitol. In some embodiments of the invention, the non-DC sweetener particles comprise isomalt. In some embodiments of the invention, the non-DC sweetener particles comprise lactitol. In some embodiments of the invention, the non-DC sweetener particles comprise mannitol.

These are typically available in a non-DC form of the relevant sugar alcohol as particles which have not been preprocessed by granulation with other sugar alcohols or binders for the purpose of obtaining so-called direct compressible particles (DC) on the basis of sugar alcohol particles which are by themselves not suitable for direct compression. Such non-DC particles of sugar alcohol may typically consist of the sugar alcohol. Therefore, non-DC sugar alcohol particles may typically be particles consisting of sugar alcohol, which is non-directly compressible in its pure form.

It should be noted that the terminology non-DC is easily understood within the field of technology. Suppliers or sugar alcohol provides clear guidance to the user as for the ability for use in relation to compression of tablets. A non-DC particle in this connection is referred to as a particle which is not expressly recommended by the supplier for compression. Examples of a non-DC grade of erythritol includes Zerose (TM) erythritol 16952F supplied by Cargill. Further examples of non-DC sugar alcohol particles include non-DC xylitol as Xivia C from Dupont, non-DC isomalt as Isomalt GS from Beneo Palatinit, non-DC mannitol as C*PharmMannidex 16700 from Cargill, non DC maltitol as Maltisorb P200 from Roquette.

Non-direct compressible (non-DC) sugar alcohols may include non-DC grades of Xylitol, non-DC grades of Erythritol, non-DC grades of Mannitol, non-DC grades of maltitol, non-DC grades of Lactitol, non-DC grades of Isomalt, or other suitable non-DC grades of sugar alcohols.

The present invention benefits from a synergy between the non-DC sugar alcohol particles and the intragranular portion. The non-DC sugar alcohol particles serves as a means for salivation which is both attractive to the user and also serves for the purpose of dissolving the intragranular portion when the tablet is chewed as fast as possible combined with an improved content of uniformity of the intragranular portion.

In some embodiments of the invention, the non-DC sweetener particles of the extragranular portion comprise one or more non-DC saccharide particles.

In some embodiments of the invention, the non-DC sweetener particles of the extragranular portion are selected from the group consisting of non-DC particles of dextrose, saccharose, fructose, and combinations thereof.

In some embodiments of the invention, the non-DC sweetener particles of the extragranular portion are selected from the group consisting of non-DC particles of dextrose, saccharose, lactose, fructose, and combinations thereof.

In some embodiments of the invention, the non-DC sweetener particles of the extragranular portion comprise non-DC dextrose particles. In some embodiments of the invention, the non-DC sweetener particles of the extragranular portion comprise non-DC fructose particles. In some embodiments of the invention, the non-DC sweetener particles of the extragranular portion comprise non-DC saccharose particles.

In some embodiments of the invention, the non-DC sweetener particles having not been granulated prior to tableting and providing the oral tablet with a plurality of discrete non-DC areas are evenly distributed in the oral tablet among the plurality of granules of the intragranular portion.

One advantage of the above embodiment may be that the even distribution of the non-DC areas promotes an effective disintegration of the module upon mastication, e.g. due to lower mechanical strength contribution from the non-DC particles, thereby facilitating effective contacting of the resulting mastication fragments formed by the mastication with saliva, again increasing dissolving of the tablet. Also, the even distribution of the non-DC areas promotes a high number of mastication fragments with non-DC sugar alcohols, which again effectively promotes salivation. Thus, a synergy between utilization of non-DC sugar alcohol particles as a disintegration promoter due to the lower mechanical strength and also as a salivation promoter in combination with the even distribution to facilitate effect dispersion of mastication fragments in the oral cavity upon mastication.

In some embodiments of the invention, the extragranular portion further comprises directly compressible (DC) sweetener particles.

One advantage of this embodiment is a surprisingly strong saliva generation compared to conventional chewable tablets and lozenges. Particularly, the plurality of non-DC areas provided in the tablet surprisingly induces a remarkable generation of saliva. Having a combination of non-DC particles and DC particles in the population of particles may further facilitate sufficient mechanical strength combined with stability of the tablet, disintegrability of the tablet upon chewing, and induced saliva generation upon chewing.

One unexpected advantage over the prior art is that the saliva generation is surprisingly sustained even after a user has swallowed the bulk-portion of the non-DC sweeteners. This sustaining of the salivation generation may be advantageous in relation to many applications of an oral tablet ranging from mouthfeel, taste, flavor perception, etc.

The term “DC sweetener particles” refer to particles of direct compressible (DC) sweeteners. It is noted that the terms “DC sweetener particles” and “DC particles” are used interchangeably. DC sweetener particles may be obtained e.g. as particles of sweetener being DC grade by nature, e.g. sorbitol, or by granulating non-DC sweetener with e.g. other sugar alcohols or binders for the purpose of obtaining so-called direct compressible particles (DC). Also, granulation of non-DC sweetener with water as binder is considered to result in “DC sweetener particles” in the present context.

In some embodiments of the invention, the DC sweetener particles of the extragranular portion is present in an amount of at least 10% by weight of the oral tablet. In some embodiments of the invention, the DC sweetener particles of the extragranular portion is present in an amount of at least 20% by weight of the oral tablet. In some embodiments of the invention, the DC sweetener particles of the extragranular portion is present in an amount of at least 30% by weight of the extragranular portion. In some embodiments of the invention, the DC sweetener particles of the extragranular portion is present in an amount of at least 40% by weight of the oral tablet.

In some embodiments of the invention, the DC sweetener particles of the extragranular portion is present in an amount of 10-70% by weight of the oral tablet. In some embodiments of the invention, the DC sweetener particles of the extragranular portion is present in an amount of 10-60% by weight of the oral tablet. In some embodiments of the invention, the DC sweetener particles of the extragranular portion is present in an amount of 10-40% by weight of the oral tablet. In some embodiments of the invention, the DC sweetener particles of the extragranular portion is present in an amount of 20-70% by weight of the oral tablet. In some embodiments of the invention, the DC sweetener particles of the extragranular portion is present in an amount of 20-60% by weight of the oral tablet. In some embodiments of the invention, the DC sweetener particles of the extragranular portion is present in an amount of 30-70% by weight of the oral tablet. In some embodiments of the invention, the DC sweetener particles of the extragranular portion is present in an amount of 30-60% by weight of the oral tablet.

In some embodiments of the invention, the DC sweetener particles of the extragranular portion comprise one or more DC sugar alcohol particles.

Examples of a direct compressible (DC) grade of erythritol include Zerose™ DC 16966 also supplied by Cargill. Further examples of DC sugar alcohols include sorbitol particles provided as e.g. Neosorb® P 300 C from Roquette, mannitol particles provided as e.g. Pearlitol® 300DC or Pearlitol 200 SD from Roquette, maltitol provided as e.g. SweetPearl® P 300 DC. xylitol provided as e.g. Xylisorb® Xtab 240 from Roquetteor Xylitab 200 from Dupont.

Direct compressible (DC) sugar alcohols may include sorbitol which is DC by nature, DC grades of Xylitol, DC grades of Erythritol, DC grades of Mannitol, DC grades of maltitol, DC grades of Lactitol, Isomalt or other suitable DC grades of sugar alcohols.

The DC embodiment benefits from a synergy between the non-DC sugar alcohol particles and the DC sugar alcohol particles. The DC sugar alcohols may be e.g. sorbitol which is direct compressible by nature or it may be other sugar alcohols which has been preprocessed, e.g. by granulation with a suitable binder, to obtain particles which when compressed may encapsulate the non-DC sugar alcohol particles into a mechanically stable tablet. At the same time the non-DC sugar alcohol particles serves as a means for salivation which is both attractive to the user and also serves for the purpose of dissolving the DC sugar alcohol particles when the tablet is chewed as fast as possible.

In an embodiment of the invention, said DC sugar alcohol particles comprises sugar alcohols selected from DC particles of sorbitol, erythritol, xylitol, lactitol, maltitol, mannitol, isomalt, and combinations thereof.

Sorbitol is an example of a sugar alcohol, which is considered DC grade, when provided as particles consisting of sorbitol, i.e. in its pure form. On the other hand, several other sugar alcohols are considered non-DC grade if providing them as particles consisting of the specific sugar alcohol. Therefore, such non-DC sugar alcohols are conventionally processed into DC grade sugar alcohols, e.g. by granulating them with e.g. a binder.

Examples of trade grades of DC sugar alcohols include sorbitol particles provided as e.g. Neosorb® P 300 C from Roquette, mannitol particles provided as e.g. Pearlitol® 300DC or Pearlitol 200 SD from Roquette, maltitol provided as e.g. SweetPearl® P 300 DC, xylitol provided as e.g. Xylisorb® Xtab 240 from Roquette or Xylitab 200 from Dupont.

In some embodiments of the invention, the DC sweetener particles of the extragranular portion comprise one or more DC saccharide particles.

In some embodiments of the invention, the weight ratio between non-DC sugar alcohol particles and DC sugar alcohol particles of the extragranular portion is between 0.2 and 1.2.

In some embodiments of the invention, the weight ratio between non-DC sugar alcohol particles and DC sugar alcohol particles of the extragranular portion is between 0.3 and 0.7.

In an embodiment of the invention, the tablet has a weight ratio between said non-DC sugar alcohol particles and said DC sugar alcohol particles, which is between 0.3 and 1.0.

The weight ratio between non-DC sugar alcohol particles and DC sugar alcohol particles have proven significant according to an embodiment of the invention in the sense that a relatively high amount of non-DC sugar alcohol particles must be present in order to obtain the mouthfeel and taste obtained through the invention. However, this taste and mouthfeel also resides in the DC sugar alcohol particles. An example of such DC sugar alcohol particle is DC grade xylitol, which, together with the non-DC sugar alcohol particles may provide a mouthfeel which is unique and very attractive to test panels.

The weight ratio between non-DC sugar alcohol particles and DC sugar alcohol particles have proven significant as mentioned above in relation to the direct sensation and mouthfeel experienced by the user but is has moreover addressed the challenge in relation to mouthfeel when DC sugar alcohol particles crumbles during the initial chew. The mechanical stability of the tablet is much desired when the tablet is in its non-chewed form, but a fast disintegration and dissolving is desirable when the tablet is chewed due to the fact that user of the tablet dislike a sandy mouthfeel induced through small hard-pressed crumbles of DC sugar alcohol. The use of a very high amount of non-DC sugar alcohol particles will facilitate a perceived fast dissolving and disintegration of the tablet after the initial chews.

According to an embodiment of the invention the tablet has a weight ratio between said non-DC sugar alcohol particles and said DC sugar alcohol particles, which is greater than 0.3, such as greater than 0.4, such as greater than 0.5.

According to an embodiment of the invention the tablet has a weight ratio between said non-DC sugar alcohol particles and said DC sugar alcohol particles, which is smaller than 0.7, such as smaller than 0.6, such as smaller than 0.55.

The weight ratio between non-DC sugar alcohol particles and DC sugar alcohol particles is important for the purpose of obtaining an advantageous taste and mouthfeel. By having an upper limit of this weight ratio, the chewer will moreover also experience a desirable crunch sensation when masticating the tablet, the crunch being obtained through the use of substantial amounts of DC sugar alcohol particles and the non-DC sugar alcohol particles.

In some embodiments of the invention, the non-DC sweetener particles having not been granulated prior to tableting and providing the oral tablet with a plurality of discrete non-DC areas are evenly distributed in at least one module of the oral tablet among DC sugar alcohol particles of the extragranular portion.

In some embodiments of the invention, saliva generation upon mastication of the oral tablet is increased compared to an oral tablet without non-DC sugar alcohol particles.

In some embodiments of the invention, the oral tablet comprises at least 30% by weight of non-DC sugar alcohol particles in the extragranular portion having a particle size above 500 μm. In an embodiment of the invention, at least 40% by weight of the non-DC sugar alcohol particles have a particle size above 500 μm. In some embodiments of the invention, at least 20% by weight of said population of particles of the extragranular portion have a particle size above 500 μm.

To the surprise of the inventor, it was seen that larger non-DC sugar alcohol particles were particularly beneficial according to the invention. In particular, larger non-DC sugar alcohol particles were seen to result in induced saliva generation, e.g. a higher total weight of saliva generated compared to smaller non-DC particles. Also, the perceived watering effect may be increased compared to smaller non-DC particles. These findings were not expected by the inventor.

In some embodiments of the invention, the extragranular portion further comprises flavor particles.

In the present context, flavor particles are to be understood as solid flavor particles. These solid flavor particles may comprise flavor bound to a carrier, such as powdered flavors containing up to 20% of flavor where a carrier is present.

Usable flavors include almond, almond amaretto, apple, Bavarian cream, black cherry, black sesame seed, blueberry, brown sugar, bubblegum, butterscotch, cappuccino, caramel, caramel cappuccino, cheesecake (graham crust), chili, cinnamon redhots, cotton candy, circus cotton candy, clove, coconut, coffee, clear coffee, double chocolate, energy cow, ginger, glutamate, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry kiwi, tropical punch, tutti frutti, vanilla, or any combination thereof.

In some embodiments of the invention, the flavor particles of the extragranular portion is present in an amount of 1-10% by weight of the oral tablet. In some embodiments of the invention, the flavor particles of the extragranular portion is present in an amount of 1-8% by weight of the oral tablet. In some embodiments of the invention, the flavor particles of the extragranular portion is present in an amount of 1-6% by weight of the oral tablet. In some embodiments of the invention, the flavor particles of the extragranular portion is present in an amount of 1-5% by weight of the oral tablet. In some embodiments of the invention, the flavor particles of the extragranular portion is present in an amount of 1-3% by weight of the oral tablet.

In some embodiments of the invention, the non-DC sweetener particles having not been granulated prior to tableting and providing the oral tablet with a plurality of discrete non-DC areas are evenly distributed in at least one module of the oral tablet among the flavor particles of the extragranular portion.

In some embodiments of the invention, the one or more active pharmaceutical ingredients comprises one or more low dose active pharmaceutical ingredients.

In some embodiments of the invention, the one or more active pharmaceutical ingredients comprises one or more low dose active pharmaceutical ingredients in an amount of less than 5% by weight of the plurality of granules.

In some embodiments of the invention, the one or more active pharmaceutical ingredients comprises one or more low dose active pharmaceutical ingredients in an amount of less than 2% by weight of the plurality of granules.

In some embodiments of the invention, the one or more active pharmaceutical ingredients comprises one or more low dose active pharmaceutical ingredients in an amount of less than 10% by weight of the oral tablet.

In some embodiments of the invention, the one or more active pharmaceutical ingredients comprises one or more low dose active pharmaceutical ingredients in an amount of less than 5% by weight of the oral tablet.

In some embodiments of the invention, the one or more active pharmaceutical ingredients comprises one or more low dose active pharmaceutical ingredients in an amount of less than 4% by weight of the oral tablet.

In some embodiments of the invention, the one or more active pharmaceutical ingredients comprises one or more low dose active pharmaceutical ingredients in an amount of less than 3% by weight of the oral tablet.

In some embodiments of the invention, the one or more active pharmaceutical ingredients comprises one or more low dose active pharmaceutical ingredients in an amount of less than 2% by weight of the oral tablet.

In some embodiments of the invention, the one or more active pharmaceutical ingredients comprises one or more low dose active pharmaceutical ingredients in an amount of less than 1% by weight of the oral tablet.

In some embodiments of the invention, a series of at least 5 samples each having the same fixed weight in the range of 0.5-2 g of the plurality of granules comprises the one or more low dose active pharmaceutical ingredients in an amount varying with a relative standard deviation (RSD) below 10%.

Generally, the method used for content uniformity of samples is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

When attempting to obtain a high degree of even distribution, insufficient mixing may lead to uneven distribution, such as undesirable agglomeration of particles within certain parts of the tablet. Also, even if mixing very thoroughly the ingredients, an undesirable handling of the mixture from the mixing to a tableting machine may lead to segregation. For example, smaller particles may typically segregate to the bottom part of a container, thereby leading to different particle distributions for different tablets. Particularly when the different ingredients have different particle sizes, e.g. if non-DC particles have a larger particle size compared to other ingredients, segregation may lead to different contents of non-DC sugar alcohols in different tablets. Yet, another aspect is that even storing a thoroughly mixed composition for too long may lead to segregation.

In is noted that the term segregation as used herein would be known to the skilled person to mean the separation of a mixture according to similarity, typically size. This may in the present context be a problem when handling a mixture comprising very different sizes of particles, e.g. in a hopper for holding and feeding the composition via a feeding mechanism to a die cavity.

In some embodiments of the invention, a series of at least 5 samples each having the same fixed weight in the range of 0.5-2 g of the plurality of granules comprises the one or more low dose active pharmaceutical ingredients in an amount varying with a relative standard deviation (RSD) below 5%.

In some embodiments of the invention, the one or more active pharmaceutical ingredients comprises one or more low dose active pharmaceutical ingredients selected from the group consisting of diphenhydramine, cetirizine, loratadine, chlorpheniramine maleate, levocetirizine, meclizine, dextromethorphan, phenylephrine, famotidine, omeprazole, doxylamine succinate, melatonin, and any combination and mixture thereof.

In some embodiments of the invention, the one or more active pharmaceutical ingredients comprises diphenhydramine. In some embodiments of the invention, the one or more active pharmaceutical ingredients comprises cetirizine. In some embodiments of the invention, the one or more active pharmaceutical ingredients comprises loratadine. In some embodiments of the invention, the one or more active pharmaceutical ingredients comprises chlorpheniramine maleate. In some embodiments of the invention, the one or more active pharmaceutical ingredients comprises chlorpheniramine levocetirizine. In some embodiments of the invention, the one or more active pharmaceutical ingredients comprises meclizine. In some embodiments of the invention, the one or more active pharmaceutical ingredients comprises dextromethorphan. In some embodiments of the invention, the one or more active pharmaceutical ingredients comprises phenylephrine. In some embodiments of the invention, the one or more active pharmaceutical ingredients comprises famotidine. In some embodiments of the invention, the one or more active pharmaceutical ingredients comprises omeprazole. In some embodiments of the invention, the one or more active pharmaceutical ingredients comprises doxylamine succinate. In some embodiments of the invention, the one or more active pharmaceutical ingredients comprises melatonin.

In some embodiments of the invention, the one or more active pharmaceutical ingredients comprises one or more low dose antihistamine ingredients selected from the group consisting of diphenhydramine, cetirizine, loratadine, chlorpheniramine maleate, levocetirizine, and any combination and mixture thereof.

In some embodiments of the invention, the one or more active pharmaceutical ingredients comprises one or more low dose cough suppressant ingredients comprising dextromethorphan.

In some embodiments of the invention, the one or more active pharmaceutical ingredients comprises one or more low dose motion sickness ingredients comprising meclizine.

In some embodiments of the invention, the one or more active pharmaceutical ingredients comprises one or more low dose decongestant ingredients comprising phenylephrine.

In some embodiments of the invention, the one or more active pharmaceutical ingredients comprises one or more low dose heartburn ingredients selected from the group consisting of famotidine, omeprazole, and any combination and mixture thereof.

In some embodiments of the invention, the one or more active pharmaceutical ingredients comprises one or more low dose insomnia ingredients selected from the group consisting of doxylamine succinate, melatonin, and any combination and mixture thereof.

In some embodiments of the invention, the one or more active pharmaceutical ingredients comprises one or more low dose active pharmaceutical ingredients present in the oral tablet in an amount of 2-30 mg. In some embodiments of the invention, the one or more active pharmaceutical ingredients comprises one or more low dose active pharmaceutical ingredients present in the oral tablet in an amount of 2-20 mg. In some embodiments of the invention, the one or more active pharmaceutical ingredients comprises one or more low dose active pharmaceutical ingredients present in the oral tablet in an amount of 2-10 mg.

In some embodiments of the invention, the one or more binders of the intragranular portion is present in an amount of 0.1 to 15% by weight of the intragranular portion.

In some embodiments of the invention, the one or more binders of the intragranular portion is present in an amount of 2 to 10% by weight of the intragranular portion.

In some embodiments of the invention, the one or more binders of the intragranular portion is selected from the group consisting of hydroxypropyl cellulose (HPC), hydroxypropylmethyl cellulose (HPMC), carboxymethyl cellulose (CMC), low substituted hydroxypropyl cellulose (L-HPC), polyvinylpyrrolidone (PVP), copovidone, and any mixture and combination thereof.

In some embodiments of the invention, the one or more excipients comprising one or more sweeteners of the intragranular portion is present in an amount of at least 30% by weight of the plurality of granules.

In some embodiments of the invention, the one or more excipients comprising one or more sweeteners of the intragranular portion is present in an amount of at least 40% by weight of the plurality of granules.

In some embodiments of the invention, the one or more excipients comprising one or more sweeteners of the intragranular portion is present in an amount of at least 50% by weight of the plurality of granules.

In some embodiments of the invention, the one or more excipients comprising one or more sweeteners of the intragranular portion is present in an amount of 40 to 60% by weight of the plurality of granules.

In some embodiments of the invention, the one or more excipients comprising one or more sweeteners of the intragranular portion comprises one or more sugar alcohols

In some embodiments of the invention, the one or more excipients comprising one or more sweeteners of the intragranular portion is selected from the group consisting of erythritol, maltitol, xylitol, isomalt, lactitol, mannitol, sorbitol and combinations thereof.

In some embodiments of the invention, the one or more excipients comprising one or more sweeteners of the intragranular portion comprises erythritol.

In some embodiments of the invention, the one or more excipients comprising one or more sweeteners of the intragranular portion comprises one or more saccharides.

In some embodiments of the invention, the one or more excipients comprising one or more sweeteners of the intragranular portion is selected from the group consisting of lactose, dextrose, saccharose, dextrin, trehalose, fructose, and combinations thereof.

In some embodiments of the invention, the one or more excipients comprising one or more sweeteners of the intragranular portion comprises dextrose.

In some embodiments of the invention, the one or more active pharmaceutical ingredients comprises one or more medium dose active pharmaceutical ingredients.

In some embodiments of the invention, the one or more active pharmaceutical ingredients comprises one or more medium dose active pharmaceutical ingredients in an amount of 5 to 40% by weight of the plurality of granules.

In some embodiments of the invention, the one or more active pharmaceutical ingredients comprises one or more medium dose active pharmaceutical ingredients in an amount of 5 to 20% by weight of the plurality of granules.

In some embodiments of the invention, the one or more active pharmaceutical ingredients comprises one or more medium dose active pharmaceutical ingredients in an amount of 4 to 30% by weight of the oral tablet.

In some embodiments of the invention, the one or more active pharmaceutical ingredients comprises one or more medium dose active pharmaceutical ingredients in an amount of 2 to 20% by weight of the oral tablet.

In some embodiments of the invention, the one or more active pharmaceutical ingredients comprises one or more medium dose active pharmaceutical ingredients in an amount of more than 2% by weight of the oral tablet. In some embodiments of the invention, the one or more active pharmaceutical ingredients comprises one or more medium dose active pharmaceutical ingredients in an amount of more than 3% by weight of the oral tablet. In some embodiments of the invention, the one or more active pharmaceutical ingredients comprises one or more medium dose active pharmaceutical ingredients in an amount of more than 4% by weight of the oral tablet. In some embodiments of the invention, the one or more active pharmaceutical ingredients comprises one or more medium dose active pharmaceutical ingredients in an amount of more than 5% by weight of the oral tablet.

In some embodiments of the invention, the one or more active pharmaceutical ingredients comprises one or more medium dose active pharmaceutical ingredients in an amount of less than 30% by weight of the oral tablet. In some embodiments of the invention, the one or more active pharmaceutical ingredients comprises one or more medium dose active pharmaceutical ingredients in an amount of less than 25% by weight of the oral tablet. In some embodiments of the invention, the one or more active pharmaceutical ingredients comprises one or more medium dose active pharmaceutical ingredients in an amount of less than 20% by weight of the oral tablet. In some embodiments of the invention, the one or more active pharmaceutical ingredients comprises one or more medium dose active pharmaceutical ingredients in an amount of less than 15% by weight of the oral tablet.

In some embodiments of the invention, a series of at least 5 samples each having the same fixed weight in the range of 0.5-2 g of the plurality of granules comprises the one or more medium dose active pharmaceutical ingredients in an amount varying with a relative standard deviation (RSD) below 10%.

Generally, the method used for content uniformity of samples is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

In some embodiments of the invention, a series of at least 5 samples each having the same fixed weight in the range of 0.5-2 g of the plurality of granules comprises the one or more medium dose active pharmaceutical ingredients in an amount varying with a relative standard deviation (RSD) below 5%.

In some embodiments of the invention, the one or more active pharmaceutical ingredients comprises one or more medium dose active pharmaceutical ingredients selected from the group consisting of diphenhydramine, fexofenadine, dimenhydrinate, meclizine, pseudoephedrine, aspirin, caffeine, theanine, and any combination and mixture thereof.

In some embodiments of the invention, the one or more active pharmaceutical ingredients comprises diphenhydramine. In some embodiments of the invention, the one or more active pharmaceutical ingredients comprises fexofenadine. In some embodiments of the invention, the one or more active pharmaceutical ingredients comprises dimenhydrinate. In some embodiments of the invention, the one or more active pharmaceutical ingredients comprises meclizine. In some embodiments of the invention, the one or more active pharmaceutical ingredients comprises pseudoephedrine. In some embodiments of the invention, the one or more active pharmaceutical ingredients comprises aspirin. In some embodiments of the invention, the one or more active pharmaceutical ingredients comprises caffeine. In some embodiments of the invention, the one or more active pharmaceutical ingredients comprises theanine.

In some embodiments of the invention, the one or more active pharmaceutical ingredients comprises one or more medium dose antihistamine ingredients selected from the group consisting of diphenhydramine, fexofenadine, and any combination and mixture thereof.

In some embodiments of the invention, the one or more active pharmaceutical ingredients comprises one or more medium dose motion sickness ingredients selected from the group consisting of dimenhydrinate, meclizine, and any combination and mixture thereof.

In some embodiments of the invention, the one or more active pharmaceutical ingredients comprises one or more medium dose decongestant ingredients comprising pseudoephedrine.

In some embodiments of the invention, the one or more active pharmaceutical ingredients comprises one or more medium dose analgesics comprising aspirin.

In some embodiments of the invention, the one or more active pharmaceutical ingredients comprises one or more medium dose alertness ingredients selected from the group consisting of caffeine, theanine, and any combination and mixture thereof.

In some embodiments of the invention, the one or more active pharmaceutical ingredients comprises one or more medium dose active pharmaceutical ingredients present in the oral tablet in an amount of 20-150 mg.

In some embodiments of the invention, the one or more active pharmaceutical ingredients comprises one or more medium dose active pharmaceutical ingredients present in the oral tablet in an amount of 30-100 mg.

In some embodiments of the invention, the one or more active pharmaceutical ingredients comprises one or more high dose active pharmaceutical ingredients.

In some embodiments of the invention, the one or more active pharmaceutical ingredients comprises one or more high dose active pharmaceutical ingredients in an amount of 20 to 60% by weight of the plurality of granules.

In some embodiments of the invention, the one or more active pharmaceutical ingredients comprises one or more high dose active pharmaceutical ingredients in an amount of 30 to 50% by weight of the plurality of granules.

In some embodiments of the invention, the one or more active pharmaceutical ingredients comprises one or more high dose active pharmaceutical ingredients in an amount of 20 to 60% by weight of the oral tablet.

In some embodiments of the invention, the one or more active pharmaceutical ingredients comprises one or more high dose active pharmaceutical ingredients in an amount of 20 to 50% by weight of the oral tablet.

In some embodiments of the invention, a series of at least 5 samples each having the same fixed weight in the range of 0.5-2 g of the plurality of granules comprises the one or more high dose active pharmaceutical ingredients in an amount varying with a relative standard deviation (RSD) below 10%.

Generally, the method used for content uniformity of samples is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

In some embodiments of the invention, a series of at least 5 samples each having the same fixed weight in the range of 0.5-2 g of the plurality of granules comprises the one or more high dose active pharmaceutical ingredients in an amount varying with a relative standard deviation (RSD) below 5%.

In some embodiments of the invention, the one or more active pharmaceutical ingredients comprises one or more high dose active pharmaceutical ingredients selected from the group consisting of fexofenadine, guaifenesin, calcium carbonate, magnesium hydroxide, acetaminophen, aspirin, ibuprofen, naproxen sodium, caffeine, and any combination and mixture thereof.

In some embodiments of the invention, the one or more active pharmaceutical ingredients comprises fexofenadine. In some embodiments of the invention, the one or more active pharmaceutical ingredients comprises fexofenadine. In some embodiments of the invention, the one or more active pharmaceutical ingredients comprises fexofenadine. In some embodiments of the invention, the one or more active pharmaceutical ingredients comprises guaifenesin. In some embodiments of the invention, the one or more active pharmaceutical ingredients comprises calcium carbonate. In some embodiments of the invention, the one or more active pharmaceutical ingredients comprises magnesium hydroxide. In some embodiments of the invention, the one or more active pharmaceutical ingredients comprises acetaminophen. In some embodiments of the invention, the one or more active pharmaceutical ingredients comprises aspirin. In some embodiments of the invention, the one or more active pharmaceutical ingredients comprises ibuprofen. In some embodiments of the invention, the one or more active pharmaceutical ingredients comprises naproxen sodium. In some embodiments of the invention, the one or more active pharmaceutical ingredients comprises caffeine.

In some embodiments of the invention, the one or more active pharmaceutical ingredients comprises one or more high dose antihistamine ingredients comprising fexofenadine.

In some embodiments of the invention, the one or more active pharmaceutical ingredients comprises one or more high dose expectorant ingredients comprising guaifenesin.

In some embodiments of the invention, the one or more active pharmaceutical ingredients comprises one or more high dose antacid ingredients selected from the group consisting of calcium carbonate, magnesium hydroxide, and any combination and mixture thereof.

In some embodiments of the invention, the one or more active pharmaceutical ingredients comprises one or more high dose analgesics ingredients selected from the group consisting of acetaminophen, aspirin, ibuprofen, naproxen sodium, and any combination and mixture thereof.

In some embodiments of the invention, the one or more active pharmaceutical ingredients comprises one or more high dose alertness ingredients comprising caffeine.

In some embodiments of the invention, the one or more active pharmaceutical ingredients comprises one or more high dose active pharmaceutical ingredients present in the oral tablet in an amount of 150-1000 mg.

In some embodiments of the invention, the one or more active pharmaceutical ingredients comprises one or more high dose active pharmaceutical ingredients present in the oral tablet in an amount of 250-600 mg.

In some embodiments of the invention, the one or more active pharmaceutical ingredients comprises two or more active pharmaceutical ingredients.

In some embodiments of the invention, the one or more active pharmaceutical ingredients comprises three or more active pharmaceutical ingredients.

In some embodiments of the invention, the one or more active pharmaceutical ingredients comprises three or more active pharmaceutical ingredients comprises acetaminophen, phenyl ephrine, and dextromethorphan.

In some embodiments of the invention, the one or more active pharmaceutical ingredients comprises three or more active pharmaceutical ingredients comprises acetaminophen, dextromethorphan, and caffeine.

In some embodiments of the invention, the one or more active pharmaceutical ingredients comprises three or more active pharmaceutical ingredients comprises acetaminophen, dextromethorphan, and guaifenisine.

In some embodiments of the invention, the one or more active pharmaceutical ingredients comprises three or more active pharmaceutical ingredients comprises acetaminophen, phenylephrine, and guaifenisine.

In some embodiments of the invention, the one or more active pharmaceutical ingredients comprises three or more active pharmaceutical ingredients comprises acetaminophen, phenylephrine, guaifenisine, and dextromethorphan.

In some embodiments of the invention, the one or more active pharmaceutical ingredients comprises a combination of two or more low dose active pharmaceutical ingredients.

In some embodiments of the invention, the one or more active pharmaceutical ingredients comprises a combination of two or more medium dose active pharmaceutical ingredients.

In some embodiments of the invention, the one or more active pharmaceutical ingredients comprises a combination of two or more high dose active pharmaceutical ingredients.

In some embodiments of the invention, the one or more active pharmaceutical ingredients comprises a combination of one or more low and medium dose active pharmaceutical ingredients.

In some embodiments of the invention, the one or more active pharmaceutical ingredients comprises a combination of one or more low and high dose active pharmaceutical ingredients.

In some embodiments of the invention, the one or more active pharmaceutical ingredients comprises a combination of one or more low, medium, and high dose active pharmaceutical ingredients.

In some embodiments of the invention, the intragranular portion further comprises a granulation diluent.

In some embodiments of the invention, the intragranular portion further comprises a granulation diluent comprising microcrystalline cellulose.

In some embodiments of the invention, the intragranular portion further comprises a granulation diluent comprising starch, such as pregelatinized starch. Starch may be supplied as Starch 1500® Partially Pregelatinized Maize Starch” from Colorcon.

In some embodiments of the invention, the intragranular portion further comprises a granulation diluent comprising microcrystalline cellulose and starch, such as pregelatinized starch.

In some embodiments of the invention, the intragranular portion further comprises a granulation diluent in an amount of at least 20% by weight of the intragranular portion.

In some embodiments of the invention, the intragranular portion further comprises a disintegrant.

In some embodiments of the invention, the intragranular portion further comprises a disintegrant in an amount of 1-10% by weight of the plurality of granules. In some embodiments of the invention, the intragranular portion further comprises a disintegrant in an amount of 1-10% by weight of the tablet.

In some embodiments of the invention, the intragranular portion further comprises a disintegrant selected from the group consisting of sodium croscarmellose, crospovidone, sodium starch glycolate, and combinations thereof.

In some embodiments of the invention, the oral tablet comprises at least two modules comprising a first tablet module and a second tablet module.

In some embodiments of the invention, the oral tablet comprises at least two modules comprising a first tablet module and a second tablet module, and wherein the extragranular portion is comprised in the first module and the intragranular portion is comprised in the second module.

An advantage of the above embodiment may be that the second module may have a higher loading capacity for active ingredients.

In some embodiments of the invention, the oral tablet comprises at least two modules comprising a first tablet module and a second tablet module, and wherein the extragranular portion is comprised in the first module and the intragranular portion is tableted into the second module.

In some embodiments of the invention, the oral tablet comprises at least two modules comprising a first tablet module and a second tablet module, and wherein the extragranular portion is tableted into the first module and the intragranular portion is tableted into the second module.

In some embodiments of the invention, the oral tablet comprises at least two modules comprising a first tablet module and a second tablet module, and wherein the extragranular portion and the intragranular portion is tableted into the first module.

In some embodiments of the invention, the oral tablet comprises at least two modules comprising a first tablet module and a second tablet module, and wherein the extragranular portion and the intragranular portion is tableted into the first module and a further portion, preferably different portion, is tableted into the second module.

In some embodiments of the invention, the oral tablet comprises at least two modules comprising a first tablet module and a second tablet module, and wherein the extragranular portion is tableted into the first module together with DC sweetener particles, such as DC sugar alcohol particles, and the intragranular portion is tableted into the second module.

In some embodiments of the invention, a series of at least 10 tablets comprise the one or more active pharmaceutical ingredients in an amount varying with a relative standard deviation (RSD) below 5%.

Generally, the method used for content uniformity of samples is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

In some embodiments of the invention, a series of at least 10 tablets comprise the one or more active pharmaceutical ingredients in an amount varying with a relative standard deviation (RSD) below 2%.

In some embodiments of the invention, the oral tablet has a unit weight of 200 to 500 mg. In some embodiments of the invention, the oral tablet has a unit weight of 500 to 2000 mg. In some embodiments of the invention, the oral tablet has a unit weight of 700 to 2000 mg. In some embodiments of the invention, the oral tablet has a unit weight of 1000 to 2000 mg.

### DETAILED DESCRIPTION

The verb “to comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”. Additionally, the words “a” and “an” when used in the present document in connection with the word comprising or containing denote “one or more.” The expression “one or more” is intended to mean one, two, three or more.

As used herein, the term “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “%” and “percent” refers to percent by weight, unless otherwise is stated.

In the present context, the phrase “population of particles” refers to a statistical population of particles. The population of particles may be characterized by a number of different parameters, e.g. statistical parameters such as distribution of particles, average particle size, particle size distribution width, etc. The population of particles may have subpopulations.

The term “particle size” relates to the ability of the particles to move through or be retained by sieve holes of a specific size. As used herein, the term “particle size” refers to the average particle size as determined according to European Pharmacopoeia 9.1 when using test method 2.9.38 particle size distribution estimation by analytical sieving, unless otherwise specifically is mentioned.

The term “particle” or similar wording is intended to denote a single, discrete composition of solid matter, such as a granule or individual elements in powder form, having a certain size that may deviate considerable.

In the present context, the “intragranular portion” is intended to mean a component that is distinguishable from the “extragranular portion”. The “intragranular portion” is a component that is typically pre-prepared and involves granulation of the various ingredients into single granules. In the present context, the “extragranular portion” my be distributed in the tablet together with the “intragranular portion”, preferably evenly distributed among the granules of the “intragranular portion”. However, the two portions may also be present in two separate layers or modules of the oral tablet.

In an embodiment of the invention the oral tablet comprises a plurality of oral tablet modules. In the present context the application of e.g. two modules are in particular advantageous as the use of non-DC sugar alcohols by nature may result in a more fragile tablet or at least the module in which the non-DC sugar alcohols are. In other words, non-DC sugar alcohols may be present primarily in one module thereby optimizing the desired salivation and sensory experience from the module and the tablet as such whereas another module may serve as a support ensuring that the desired stability and friability of the complete tablet is obtained.

In an embodiment of the invention the plurality of modules are slice-like layers. The term “slice-like” layer is a plain but very precise way of to the skilled person how a module may be provided, as such a layer is a standard structure obtained through conventional tableting procedures.

According to an embodiment of the invention, the tablet has two modules. Optionally, a coating may be applied around the two modules to form the final tablet.

In the present context the term “release” refers to the released substance being liberated from the water-soluble matrix. In some embodiments, the process of releasing a substance corresponds to the substance being dissolved in saliva. The term “release” in the present context is intended to mean tested under “in vivo” conditions, if not stated otherwise. In the present context, when the tablet is masticated, “in vivo” conditions is intended to mean that a sample is masticated with a chewing frequency of 60 chews pr. minute for a certain period of time in a test panel of 8 test persons, if not stated otherwise. These test persons abstain from eating and drinking at least 30 minutes before initiation of any test. The test persons are healthy persons appointed on an objective basis according to specified requirements.

The term “sustained release” or “extended release” is herein intended to mean prolonged release over time. The term “rapid release” or “quick release” or “high release” is herein intended to mean a higher content released for a given period of time.

As used herein the term “oral tablet” is considered as a tablet for oral use. Particularly, the oral tablet is considered as formed by tableting, i.e. compression of a particle composition, comprising the mentioned population of particles. Thus, the tablet is considered a compressed tablet formed by a plurality of particles. Typically, the oral tablet may also be referred to as a tablet.

The term “weight of the oral tablet” or similar wording meaning the same is defined in the present context as weight of the oral tablet, not including the weight of an outer coating, such as a hard coating, soft coating, and the like.

By the phrase “texture” is meant a qualitative measure of the properties of the oral tablet and of the overall mouth-feel experienced by the user during use. Thus, the term “texture” encompasses measurable quantities such as hardness as well as more subjective parameters related to the feel experienced by a user.

The term “sustained release” or “extended release” is herein intended to mean prolonged release over time. The term “rapid release” or “quick release” or “high release” is herein intended to mean a higher content released for a given period of time. The term “controlled release” is intended to mean a release of a substance from an oral tablet by the aid of active use of the oral tablet in the oral cavity of the subject, whereby the active use is controlling the amount of substance released.

A “self-emulsifying agent” is an agent which will form an emulsion when presented with an alternate phase with a minimum energy requirement. In contrast, an emulsifying agent, as opposed to a self-emulsifying agent, is one requiring additional energy to form an emulsion.

Due to the poor solubility of certain active ingredients in physiological fluids, it is an unmet need to have a high dose of certain active ingredients in a form, that solubilize the active ingredient upon mixture with the body physiological fluids to facilitate bio-absorption. To overcome low oral bioavailability, various lipid-based drug delivery systems and self-emulsifying systems have been developed. Lipid-based delivery systems and particularly self-emulsifying drug delivery systems (SEDDS) have been demonstrated to increase the solubility, dissolution and bioavailability of many insoluble active ingredients. However, lipid-based and SEDDS delivery systems are very limited by the amount of active ingredient loading that has to be dissolved in the vehicle composition. Higher concentration of active ingredients are obtained using co-solvents, which enable loads of up to 30% in specific cases.

Particular challenges are considered to arise in formulating oral tablets with SEDDS. For instance, challenges may arise with obtaining a homogenous mixture where variations are avoided and a safe and convenient delivery may be obtained. Also, the general formulation of the oral tablets offering convenience to the user need not be compromised which is often the case if precaution is not taken, such as in cases where a high load of active ingredients is needed.

Particularly with respect to SEDDS, the formulation of the present invention may provide some clear benefits, both allowing a higher load of active ingredients and at the same time offer improved sensorics properties of the formulation during use. Other advantages are also present.

Importantly, the presence of SEDDS or at least a self-emulsifying agent was seen to act in synergy with increased saliva generation. While increased saliva generation was seen to distribute certain active ingredients and allocate a higher load of active ingredients to for instance mucosal surfaces, the presence of SEDDS or at least a self-emulsifying agent was seen to further increase the uptake of these active ingredients through oral surfaces. Accordingly, the synergy between the presence of SEDDS or at least a self-emulsifying agent and increased saliva generation according to the invention was a surprise to the inventors. In some embodiments, increased saliva generation may result in a higher exposure of the active ingredients to mucosal surfaces. The presence of SEDDS may work to increase the affinity of the active ingredients from this saliva to the mucosa. Particularly, the potential of SEDDS to have a high load of active ingredients further contributes to the synergy of the tablet according to the invention in combination with improved saliva generation.

In the present context, SEDDS is a solid or liquid dosage form comprising an oil phase, a surfactant and optionally a co-surfactant, characterized primarily in that said dosage form can form oil-in-water emulsion spontaneously in the oral cavity or at ambient temperature (referring generally to body temperature, namely 37° C.). When a SEDDS enters the oral cavity, it is initially self-emulsified as emulsion droplets and rapidly dispersed throughout the oral cavity, and thus reducing the irritation caused by the direct contact of the active ingredient with the mucous membrane of the oral cavity, and hence helping on taste-masking active ingredients. In the oral cavity, the structure of the emulsion microparticulate will be changed or destroyed. The resulting microparticulate of micrometer or nanometer level can penetrate into the mucous membrane of for instance the oral cavity, and the absorbed oil droplets enter the blood circulation, thereby significantly improving the bioavailability of the active ingredient.

In an embodiment of the invention, the self-emulsifying system comprises one or more emulsifiers and one or more oil carriers.

In an embodiment of the invention, the self-emulsifying system comprises one or more emulsifiers, one or more oil carriers and one or more solubilizers.

In an embodiment of the invention, the self-emulsifying system comprises one or more emulsifiers, one or more oil carriers, one or more solubilizers and one or more solvents.

In an embodiment of the invention, the self-emulsifying system comprises one or more emulsifiers and one or more solvents.

In an embodiment of the invention, the self-emulsifying system comprises one or more emulsifiers that have both emulsifying and solubilizing properties.

In an embodiment of the invention, the self-emulsifying system comprises one or more emulsifiers that act as both an emulsifier and a carrier.

In an embodiment of the invention, the self-emulsifying system comprises one or more emulsifiers that act as both an emulsifier, a carrier and a solubilizer.

In an embodiment of the invention, the self-emulsifying system comprises one or more fatty acids, one or more glycerols, one or more waxes, one or more flavonoids and one or more terpenes.

In an embodiment of the invention, the self-emulsifying system comprises one or more emulsifiers that have an HLB-value of more than 6, preferably of 8-18.

In an embodiment of the invention, the one or more emulsifiers are selected from the group consisting of PEG-35 castor oil, PEG-6 oleoyl glycerides, PEG-6 linoleoyl glycerides, PEG-8 caprylic/capric glyceride, sorbitan monolaurate, sorbitan monooleate, polyoxyethylene (20) sorbitan monolaurate, polyoxyethylene (60) sorbitan monostearate, polyoxyethylene (80) sorbitan monooleate, lauroylpoloxyl-32 glycerides, stearoyl polyoxyl-32 glycerides, polyoxyl-32 stearate, propylene glycol mono laurate, propylene glycol di laurate, and mixtures and combinations thereof.

In an embodiment of the invention, the one or more emulsifiers comprise PEG-35 castor oil.

In an embodiment of the invention, the oil carrier is selected from the group consisting of natural fatty acids; medium-chain triglycerides of caprylic (C8) and capric (C10) acids; propylene glycol esters of caprylic (C8) and capric (C10) acids; mono-, di- and triglycerides of mainly linoleic (C18:2) and oleic (C18:1) acids; fatty acid 18:1 cis-9; natural fatty acids; mono-, di- and triglycerides of oleic (C18:1) acid, and mixtures and combinations thereof.

In an embodiment of the invention, the one or more solvents are selected from the group consisting of polyglyceryl-3 dioleate, 1,2-propandiol, polyethylene glycol 300, polyethylene glycol 400, diethylene glycol monoethyl ether, and mixtures and combinations thereof.

In an embodiment of the invention, the oil carrier is selected from the group consisting of corn oil, Labrafac lipophile WL1349, Labrafac PG, Maisine CC, oleic acid, olive oil, Peceol, and mixtures and combinations thereof.

In an embodiment of the invention, the one or more solvents are selected from the group consisting of polyglyceryl-3 dioleate, 1,2-propandiol, polyethylene glycol 300, polyethylene glycol 400, diethylene glycol monoethyl ether, and mixtures and combinations thereof.

In an embodiment of the invention, the one or more solubilizers are selected from the group consisting of lauroylpoloxyl-32 glycerides; stearoyl polyoxyl-32 glycerides; Polyoxyl-32 stearate; synthetic copolymer of ethylene oxide (80) and propylene oxide (27); polyvinyl caprolactam-polyvinyl acetate-polyethylene glycol graft co-polymer; alpha-, beta- or gamma cyclodextrins and derivatives thereof; pea proteins (globulins, albumins, glutelins proteins); and mixtures and combinations thereof.

In an embodiment of the invention, the tablet comprises an active pharmaceutical ingredient and a self-emulsifying system that when hydrated with saliva upon oral administration forms an emulsion.

In an embodiment of the invention, the oral tablet further comprises one or more lipids.

In the following raw materials will refer to the mixed particles to be compressed into a tablet according to embodiments of the invention unless otherwise stated.

The following description outlines explanations of how the tablet of the invention may be produced and further details of what may be added to the inventive composition.

Typically, the process of manufacture of the inventive tablet may be performed in a single tablet press, such as a rotary tablet press. But it may be a benefit under some circumstances to apply a separate tablet press.

Preferably, the upper punch is convex which gives the upper face of the pressed tablet a concave form.

It should of course be noted that the shape of the punches may vary depending of the desired tablet shape.

In some embodiments of the invention, pressing of the tablets are performed at a force of 20 to 50 kN.

In one embodiment examples of active substances includes a comprehensive list of which is found e.g. in WO 00/25598, which is incorporated herein by reference, including drugs, dietary supplements, antiseptic agents, pH adjusting agents, anti-smoking agents and substances for the care or treatment of the oral cavity and the teeth such as hydrogen peroxide and compounds capable of releasing urea during chewing. Examples of useful active substances in the form of antiseptics include salts and derivatives of guanidine and biguanidine (for instance chlorhexidine diacetate) and the following types of substances with limited water-solubility: quaternary ammonium compounds (e.g. ceramine, chloroxylenol, crystal violet, chloramine), aldehydes (e.g. paraformaldehyde), derivatives of dequaline, polynoxyline, phenols (e.g. thymol, p-chlorophenol, cresol), hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. also Martindale, The Extra Pharmacopoeia, 28th edition, pages 547-578; metal salts, complexes and compounds with limited water-solubility, such as aluminum salts, (for instance aluminum potassium sulphate AIK (SO4) 2, 12H2O) and salts, complexes and compounds of boron, barium, strontium, iron, calcium, zinc, (zinc acetate, zinc chloride, zinc gluconate), copper (copper chloride, copper sulphate), lead, silver, magnesium, sodium, potassium, lithium, molybdenum, vanadium should be included; other compositions for the care of mouth and teeth: for instance; salts, complexes and compounds containing fluorine (such as sodium fluoride, sodium monofluorophosphate, aminofluorides, stannous fluoride), phosphates, carbonates and selenium. Further active substances can be found in J. Dent. Res. Vol. 28 No. 2, pages 160-171,1949.

Examples of active substances in the form of agents adjusting the pH in the oral cavity include: acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulphates or oxides of sodium, potassium, ammonium, magnesium or calcium, especially magnesium and calcium.

Active ingredients may comprise the below mentioned compounds or derivates thereof but are not limited thereto: Acetaminophen, Acetylsalicylic acid, Buprenorphine, Bromhexin, Celcoxib, Codeine, Diphenhydramineclofenac, Etoricoxib, Ibuprofen, Indometacin, Ketoprofen, Lumiracoxib, Morphine, Naproxen, Oxycodon, Parecoxib, Piroxicam, Pseudoefedrin, Rofecoxib, Tenoxicam, Tramadol, Valdecoxib, Calciumcarbonat, Magaldrate, Disulfiram, Bupropion, Nicotine, Azithromycin, Clarithromycin, Clotrimazole, Erythromycin, Tetracycline, Granisetron, Ondansetrone, Prometazin, Tropisetron, Brompheniramine, Ceterizin, leco-Ceterizin, Chlorcyclizine, Chlorpheniramin, Chlorpheniramin, Difenhydramine, Doxylamine, Fenofenadin, Guaifenesin, Loratidin, des-Loratidin, Phenyltoloxamine, Promethazin, Pyridamine, Terfenadin, Troxerutin, Methyldopa, Methylphenidate, Benzalcon. Chloride, Benzeth. Chloride, Cetylpyrid. Chloride, Chlorhexidine, Ecabet-sodium, Haloperidol, Allopurinol, Colchinine, Theophylline, Propanolol, Prednisolone, Prednisone, Fluoride, Urea, Actot, Glibenclamide, Glipizide, Metformin, Miglitol, Repaglinide, Rosiglitazone, Apomorfin, Cialis, Sildenafil, Vardenafil, Diphenoxylate, Simethicone, Cimetidine, Famotidine, Ranitidine, Ratinidine, cetrizin, Loratadine, Aspirin, Benzocaine, Dextrometorphan, Phenylpropanolamine, Pseudoephedrine, Cisapride, Domperidone, Metoclopramide, Acyclovir, Dioctylsulfosucc, Phenolphtalein, Almotriptan, Eletriptan, Ergotamine, Migea, Naratriptan, Rizatriptan, Sumatriptan, Zolmitriptan, Aluminum salts, Calcium salts, Ferro salts, Ag-salts, Zinc-salts, Amphotericin B, Chlorhexidine, Miconazole, Triamcinolonacetonid, Melatonine, Phenobarbital, Caffeine, Benzodiazepiner, Hydroxyzine, Meprobamate, Phenothiazine, Buclizine, Brometazine, Cinnarizine, Cyclizine, Difenhydramine, Dimenhydrinate, Buflomedil, Amphetamine, Caffeine, Ephedrine, Orlistat, Phenylephedrine, Phenylpropanolamin, Pseudoephedrine, Sibutramin, Ketoconazole, Nitroglycerin, Nystatin, Progesterone, Testosterone, Vitamin B12, Vitamin C, Vitamin A, Vitamin D, Vitamin E, Pilocarpin, Aluminumaminoacetat, Cimetidine, Esomeprazole, Famotidine, Lansoprazole, Magnesiumoxide, Nizatide and or Ratinidine.

The invention is suitable for increased or accelerated release of active agents selected among the group of dietary supplements, oral and dental compositions, antiseptic agents, pH adjusting agents, anti-smoking agents, sweeteners, flavorings, aroma agents or drugs. Some of those will be described below.

The active agents to be used in connection with the present invention may be any substance desired to be released from the tablet. The active agents, for which a controlled and/or accelerated rate of release is desired, are primarily substances with a limited water-solubility, typically below 10 g/100 mL inclusive of substances which are totally water-insoluble. Examples are medicines, dietary supplements, oral compositions, anti-smoking agents, highly potent sweeteners, pH adjusting agents, flavorings etc.

Other active ingredients are, for instance, paracetamol, benzocaine, cinnarizine, menthol, carvone, caffeine, chlorhexidine-di-acetate, cyclizine hydrochloride, 1,8-cineol, nandrolone, miconazole, mystatine, sodium fluoride, nicotine, cetylpyridinium chloride, other quaternary ammonium compounds, vitamin E, vitamin A, vitamin D, glibenclamide or derivatives thereof, progesterone, acetylsalicylic acid, dimenhydrinate, cyclizine, metronidazole, sodium hydrogen carbonate, the active components from ginkgo, the active components from propolis, the active components from ginseng, methadone, oil of peppermint, salicylamide, hydrocortisone or astemizole.

Examples of active agents in the form of dietary supplements are for instance salts and compounds having the nutritive effect of vitamin B2 (riboflavin), B12, folinic acid, folic acid, niacine, biotine, poorly soluble glycerophosphates, amino acids, the vitamins A, D, E and K, minerals in the form of salts, complexes and compounds containing calcium, phosphorus, magnesium, iron, zinc, copper, iodine, manganese, chromium, selenium, molybdenum, potassium, sodium or cobalt.

Furthermore, reference is made to lists of nutritionists accepted by the authorities in different countries such as for instance US code of Federal Regulations, Title 21, Section 182.5013.182 5997 and 182.8013-182.8997.

Examples of active agents in the form of antiseptics are for instance salts and compounds of guanidine and biguanidine (for instance chlorhexidine diacetate) and the following types of substances with limited water-solubility: quaternary ammonium compounds (for instance ceramine, chloroxylenol, crystal violet, chloramine), aldehydes (for instance paraformaldehyde), compounds of dequaline, polynoxyline, phenols (for instance thymol, para chlorophenol, cresol) hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. furthermore Martindale, The Extra Pharmacopoeia, 28th edition, pages 547-578; metal salts, complexes and compounds with limited water-solubility, such as aluminum salts, (for instance aluminum potassium sulphate AIK (SO4) 2,12H2O) and furthermore salts, complexes and compounds of boron, barium, strontium, iron, calcium, zinc, (zinc acetate, zinc chloride, zinc gluconate), copper (copper chloride, copper sulfate), lead, silver, magnesium, sodium, potassium, lithium, molybdenum, vanadium should be included; other compositions for the care of mouth and teeth: for instance; salts, complexes and compounds containing fluorine (such as sodium fluoride, sodiummonofluorophosphate, amino fluorides, stannous fluoride), phosphates, carbonates and selenium.

Cf. furthermore J. Dent.Res. Vol. 28 No. 2, pages 160-171, 1949, wherein a wide range of tested compounds is mentioned.

Examples of active agents in the form of agents adjusting the pH in the oral cavity include for instance: acceptable acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulfates or oxides of sodium, potassium, ammonium, magnesium or calcium, especially magnesium and calcium.

Examples of active agents in the form of anti-smoking agents include for instance: nicotine, tobacco powder or silver salts, for instance silver acetate, silver carbonate and silver nitrate.

Further examples of active agents are medicines of any type.

Examples of active agents in the form of medicines include caffeine, salicylic acid, salicyl amide and related substances (acetylsalicylic acid, choline salicylate, magnesium salicylate, sodium salicylate), paracetamol, salts of pentazocine (pentazocine hydrochloride and pentazocinelactate), buprenorphine hydrochloride, codeine hydrochloride and codeine phosphate, morphine and morphine salts (hydrochloride, sulfate, tartrate), methadone hydrochloride, ketobemidone and salts of ketobemidone (hydrochloride), beta-blockers, (propranolol), calcium antagonists, verapamil hydrochloride, nifedinpine as well as suitable substances and salts thereof mentioned in Pharm. Int., Nov.85, pages 267-271, Barney H. Hunter and Robert L. Talbert, nitroglycerine, erythrityl tetranitrate, strychnine and salts thereof, lidocaine, tetracaine hydrochloride, etorphine hydrochloride, atropine, insulin, enzymes (for instance papain, trypsin, amyloglucosidase, glucoseoxidase, streptokinase, streptodornase, dextranase, alpha amylase), polypeptides (oxytocin, gonadorelin, (LH.RH), desmopressin acetate (DDAVP), isoxsuprine hydrochloride, ergotamine compounds, chloroquine (phosphate, sulfate), isosorbide, demoxytocin, heparin.

Other active ingredients include beta-lupeol, Letigen®, Sildenafil citrate and derivatives thereof.

Further examples of active ingredients include dental products including Carbamide, CPP Caseine Phospho Peptide; Chlorhexidine, Chlorhexidine di acetate, Chlorhexidine Chloride, Chlorhexidine di gluconate, Hexetedine, Strontium chloride, Potassium Chloride, Sodium bicarbonate, Sodium carbonate, Fluor containing ingredients, Fluorides, Sodium fluoride, Aluminum fluoride.

Further examples of active ingredients include Ammonium fluoride, Calcium fluoride, Stannous fluoride, Other fluor containing ingredients Ammonium fluorosilicate, Potassium fluorosilicate, Sodium fluorosilicate, Ammonium monofluorphosphate, Calcium monofluorphosphate, Potassium monofluorphosphate, Sodium monofluorphosphate, Octadecentyl Ammonium fluoride, Stearyl Trihydroxyethyl Propylenediamine Dihydrofluoride

Further examples of active ingredients include vitamins. Vitamins include A, B1, B2, B6, B12, Folinic acid, Folic acid, niacin, Pantothenic acid, biotine, C, D, E, K. Minerals include Calcium, phosphor, magnesium, iron, Zinc, Copper, Iod, Mangan, Crom, Selene, Molybden. Other active ingredients include:

Q10®, enzymes. Natural drugs including Ginkgo Biloba, ginger, and fish oil.

Further examples of active ingredients include migraine drugs such as Serotonin antagonists: Sumatriptan, Zolmitriptan, Naratriptan, Rizatriptan, Eletriptan; nausea drugs such as Cyclizin, Cinnarizin, Dimenhydramin, Difenhydrinat; hay fever drugs such as Cetrizin, Loratidin, pain relief drugs such as Buprenorfin, Tramadol, oral disease drugs such as Miconazol, Amphotericin B, Triamcinolonaceton; and the drugs Cisaprid, Domperidon, Metoclopramid. In a preferred embodiment the invention relates to the release of Nicotine and its salts.

In an advantageous embodiment of the invention the active ingredient is selected from active ingredients for the throat selected from acetylcysteine, ambroxol, amylmetacresol, benzocaine, bisacodyl, bismuth subsalicylate, bromhexine, cetirizine, cetylpyridinium, chlorhexidine, dextromethorphan hydrobromide, 2,4-dichlorobenzyl alcohol, doxylamine succinate, eucalyptus oil, flurbiprofen, glycerin, hexylresorcinol, lidocaine, menthol, myrrh, paracetamol, pectin, peppermint oil, phenol, phenylephrine, povidone-iodine, pseudoephedrine, ranitidine, simethicone, sodium docusate, spearmint, zinc, or any combination thereof; active ingredients for the gastrointestinal tract selected from alginate, atenolol, aspirin (acetylsalicylic acid), ampicillin, aminosalicylates, anhydrous citric acid, aspirin, bisacodyl, bismuth subsalicylate, bupropion, caffeine, calcium, calcium carbonate, cetirizine, cimetidine, cisapride, clarithromycin, desloratadine, dexlansoprazole, diphenhydramine HCl, diphenhydramine citrate, dimenhydrinate, docusate erythromycin, dopamine, esomeprazole, famotidine, fexofenadine HCl, guaifenesin, hydrotalcite, ibuprofen, ketoprofen, lactase enzyme, lansoprazole, loratadine, lorcaserin, loperamide, loperamide HCl, magnesium, magnesium carbonate, magnesium hydroxide, melatonin, methamphetamine HCl, metoclopramide, metronidazole, montelukast, mycostatin, naltrexone, naproxen, naproxen sodium, nizatidine, omeprazole, ondansetron, orlistat, pantoprazole, paracetamol (acetaminophen), pectin, phentermine HCl, polypodium leucotomos, prednisolone, prednisone, progesterone, propranolol, propantheline bromide, pseudoephedrine HCl, phentermine, rabeprazole, ranitidine, roflumilast, scopoloamine butyl hydroxide, simethicone, sodium, sodium bicarbonate, sodium docusate, sumatriptan, testosterone, tetracycline, topiramate, vitamin A, vitamin B, vitamin B12, vitamin C (ascorbic acid), vitamin D, and vitamin E, vitamin K, or any combination thereof, and active ingredients for buccal absorption selected from atenolol, baclofen, caffeine, carvedilol, chlorpheniramine, chlorpheniramine maleate, fluticasone propionate, maleate, desmopressin, diltiazem hydrochloride, doxylamine succinate, mycostatin, nicotine, nifedipine, nitroglycerin, omeprazole, ondansetron, oxymetazoline HCl, oxytocin, phenylephrine, piroxicam, prednisone, propranolol, salbutamol sulphate, scopoloamine butyl hydroxide, sumatriptan, triamcinolonacetonid, and any combination thereof.

A tablet according to the invention may, if desired, include one or more fillers/texturisers including as examples, magnesium and calcium carbonate, sodium sulphate, ground limestone, silicate compounds such as magnesium and aluminum silicate, kaolin and clay, aluminum oxide, silicium oxide, talc, titanium oxide, mono-, di- and tri-calcium phosphates, cellulose polymers and combinations thereof.

In an embodiment of the invention, the formulation comprises further ingredients selected from the group consisting of flavors, dry-binders, anti-caking agents, emulsifiers, antioxidants, enhancers, mucoadhesives, absorption enhancers, high intensity sweeteners, softeners, colors, active ingredients, water-soluble indigestible polysaccharides, water-insoluble polysaccharides or any combination thereof.

According to embodiments of the invention, the emulsifiers may be selected from the group consisting of sucrose ester of fatty acids (such as sucrose mono stearate), polyethylene glycol esters or ethers (PEG) (such as caprylocaproyl macrogol-8 glycerides and lauroyl macrogol-32-glycerides), mono- and diglyceride of fatty acids (such as glycerol monostearate, glycerol monolaurate, glyceryl behenate ester), acetic acid esters of mono- and diglycerides of fatty acids (Acetem), polyoxyethylene alkyl ethers, diacetyl tartaric ester of monoglycerides, lactylated monoglycerides, glycerophospholipids (such as lecithin), poloxamer (non-ionic block copolymer of ethylene oxide and propylene oxide), cyclodextrins, fatty acid esters of sorbitol (such as sorbitan monolaurate, sorbitan monostearate, sorbitan tristearate, polysorbates).

According to embodiments of the invention, flavors may be selected from the group consisting of coconut, coffee, chocolate, vanilla, grape fruit, orange, lime, menthol, liquorice, caramel aroma, honey aroma, peanut, walnut, cashew, hazelnut, almonds, pineapple, strawberry, raspberry, tropical fruits, cherries, cinnamon, peppermint, wintergreen, spearmint, eucalyptus, and mint, fruit essence such as from apple, pear, peach, strawberry, apricot, raspberry, cherry, pineapple, and plum essence. The essential oils include peppermint, spearmint, menthol, eucalyptus, clove oil, bay oil, anise, thyme, cedar leaf oil, nutmeg, and oils of the fruits mentioned above.

The active ingredient may also be one or more cannabinoids selected from: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCV A). More preferably the one or more cannabinoid is CBD or THC.

Antioxidants suitable for use include butylated hydroxyanisole (BHA), butylated hydroxytoluene (BHT), betacarotenes, tocopherols, acidulants such as Vitamin C (ascorbic acid or corresponding salts (ascorbates)), propyl gallate, catechins, green tea extract other synthetic and natural types or mixtures thereof.

High intensity sweetening agents can also be used according to preferred embodiments of the invention. Preferred high intensity sweeteners include, but are not limited to sucralose, aspartame, salts of acesulfame, alitame, neotame, saccharin and its salts, cyclamic acid and its salts, glycyrrhizin, dihydrochalcones, thaumatin, monellin, monk fruit extract, advantame, stevioside and the like, alone or in combination.

In order to provide longer lasting sweetness and flavor perception, it may be desirable to encapsulate or otherwise control the release of at least a portion of the high intensity sweeteners.

Techniques such as wet granulation, wax granulation, spray drying, spray chilling, fluid bed coating, conservation, encapsulation in yeast cells and fiber extrusion may be used to achieve desired release characteristics. Encapsulation of sweetening agents can also be provided using another formulation component such as a resinous compound.

Usage level of the high-intensity sweetener will vary considerably and will depend on factors such as potency of the sweetener, rate of release, desired sweetness of the product, level and type of flavor used and cost considerations. Thus, the active level of artificial sweetener may vary from about 0.001 to about 8% by weight (preferably from about 0.02 to about 8% by weight). When carriers used for encapsulation are included, the usage level of the encapsulated high-intensity sweetener will be proportionately higher.

The invention, if desired, may include one or more fillers/texturizers including as examples, magnesium- and calcium carbonate, sodium sulphate, ground limestone, silicate compounds such as magnesium- and aluminum silicate, kaolin and clay, aluminum oxide, silicium oxide, talc, titanium oxide, mono-, di- and tri-calcium phosphates, cellulose polymers, such as wood, and combinations thereof. According to an embodiment of the invention, one preferred filler/texturizer is calcium carbonate.

In an embodiment of the invention, the formulation comprises particles comprising gum base, and wherein the formulation is designed to be masticated into a coherent residual containing water-insoluble components.

The application of gum may in the present context may invoke a delay of release for active ingredients and this may again promote the buccal and upper throat absorption of active pharmaceutical ingredient when this is released from the oral tablet during mastication.

The application of gum may in the present context may invoke a delay of release for active ingredients and this may again promote the buccal and upper throat absorption of active pharmaceutical ingredient when this is released from the formulation during mastication.

In an embodiment of the invention, the formulation contains particles comprising gum base, and wherein the gum base comprises at least 5% by weight of elastomer.

When including gum base in the formulation sugar alcohols typically constitute from about 5 to about 95% by weight, more typically about 20 to about 80% by weight such as 30 to 70% or 30 to 60% by weight of the formulation.

In such an embodiment of the invention, the formulation further comprises, beside the already described sweeteners, materials selected from the group consisting of bulk sweeteners, flavors, dry-binders, anti-caking agents, emulsifiers, antioxidants, enhancers, absorption enhancers, buffers, high intensity sweeteners, softeners, colors, or any combination thereof.

### EXAMPLES

### Preparation of Two-Layer Tablets

TABLE 1A
Oral tablet compositions for first layer of bi-layer tablets
containing variants of non-DC sugar alcohols. Amounts are
given in wt-% of the respective layer of the tablet.

| Raw material | | | | | | |
|---|---|---|---|---|---|---|
| (wt %) First layer | Ex1 | Ex2 | Ex3 | Ex4 | Ex5 | Ex6 |
| Non-DC Xylitol | 50 | — | — | — | — | — |
| Sorbitol** | — | 50 | — | — | — | — |
| Non-DC Isomalt | — | — | 50 | — | — | — |
| Non-DC Erythritol | — | — | — | 50 | — | — |
| Non-DC Mannitol | — | — | — | — | 50 | — |
| Non-DC | — | — | — | — | — | 50 |
| Maltitol | | | | | | |
| Intragranular | 43.75 | 43.75 | 43.75 | 43.75 | 43.75 | 43.75 |
| portion* | | | | | | |
| Flavor | 4 | 4 | 4 | 4 | 4 | 4 |
| HIS | 0.25 | 0.25 | 0.25 | 0.25 | 0.25 | 0.25 |
| Magnesium | 1 | 1 | 1 | 1 | 1 | 1 |
| Stearate | | | | | | |
| Binder HPC | 1 | 1 | 1 | 1 | 1 | 1 |

*Including a granulation of 1% phenylephrine HCl, 48% microcrystalline cellulose (MCC), 46% lactose and 5% starch (by weight of intragranular portion).
**non-granulated sorbitol.

TABLE 1B
Oral tablet compositions for first layer of bi-layer tablets
containing variants of non-DC sugars and sugar alcohols. Amounts
are given in wt-% of the respective layer of the tablet.

| Raw material | | | | | | |
|---|---|---|---|---|---|---|
| (wt %) First layer | Ex7 | Ex8 | Ex9 | Ex10 | Ex11 | Ex12 |
| Non-DC lactitol | 50 | — | — | — | — | — |
| Non-DC lactose | — | 50 | — | — | — | — |
| Non-DC fructose | — | — | 50 | — | — | — |
| Non-DC dextrose | — | — | — | 50 | — | — |
| Non-DC | — | — | — | — | 50 | — |
| saccharose | | | | | | |
| DC Isomalt | — | — | — | — | — | 44.25 |
| Intragranular | 44.25 | 44.25 | 44.25 | 44.25 | 44.25 | — |
| portion* | | | | | | |
| Non-DC | — | — | — | — | — | 50 |
| erythritol | | | | | | |
| Flavor | 4 | 4 | 4 | 4 | 4 | 4 |
| HIS | 0.25 | 0.25 | 0.25 | 0.25 | 0.25 | 0.25 |
| Magnesium | 1 | 1 | 1 | 1 | 1 | 1 |
| Stearate | | | | | | |
| Binder HPC | 0.5 | 0.5 | 0.5 | 0.5 | 0.5 | 0.5 |

*Including a granulation of 1% phenylephrine HCl, 48% microcrystalline cellulose (MCC), 46% dextrose and 5% starch (by weight of intragranular portion).

TABLE 2
Oral tablet compositions for the second layer of bi-layered tablets.
Amounts are given in wt-% of the respective layer of the tablet.

| Raw material (wt | | | |
|---|---|---|---|
| %) Second layer | Ex1-12 | Ex13-24 | Ex25-36 |
| DC Maltitol | 94.75 | — | — |
| DC Xylitol | — | 94.75 | — |
| Intragranular portion* | — | — | 94.75 |
| Flavor | 4 | 4 | 4 |
| HIS | 0.25 | 0.25 | 0.25 |
| Magnesium Stearate | 1 | 1 | 1 |

*Including a granulation of 1% phenylephrine HCl, 48% microcrystalline cellulose (MCC), 46% lactose and 5% starch (by weight of intragranular portion).

TABLE 3
Oral tablet compositions for bi-layered tablets containing
variants of intragranular portions in layer 2. Amounts are
given in wt-% of the respective layer of the tablet.

| | Ex37 | Ex37A | Ex38 | Ex 38A | Ex38B | Ex38C |
|---|---|---|---|---|---|---|
| Raw material | | | | | | |
| (wt %) First layer | | | | | | |
| Non-DC Erythritol | 50 | 50 | 50 | 50 | 50 | 50 |
| DC Isomalt | 43.75 | 43.75 | 43.75 | 43.75 | 43.75 | 43.75 |
| Flavor | 4 | 4 | 4 | 4 | 4 | 4 |
| HIS | 0.25 | 0.25 | 0.25 | 0.25 | 0.25 | 0.25 |
| Magnesium | 1 | 1 | 1 | 1 | 1 | 1 |
| Stearate | | | | | | |
| Binder HPC | 1 | 1 | 1 | 1 | 1 | 1 |
| Raw material | | | | | | |
| (wt %) Second layer | | | | | | |
| Intragranular | 94.75 | 100 | — | — | — | — |
| portion 1* | | | | | | |
| Intragranular | — | — | 94.75 | — | — | — |
| portion 2 ** | | | | | | |
| Intragranular | — | — | — | 94.75 | — | — |
| portion 3*** | | | | | | |
| Intragranular | — | — | — | — | 94.75 | 100 |
| portion 4**** | | | | | | |
| Flavor | 4 | — | 4 | 4 | 4 | — |
| HIS | 0.25 | — | 0.25 | 0.25 | 0.25 | — |
| Magnesium | 1 | — | 1 | 1 | 1 | — |
| Stearate | | | | | | |

*Including a granulation of 1% phenylephrine HCl, 48% microcrystalline cellulose (MCC), 46% dextrose and 5% starch (by weight of intragranular portion).
** Including a granulation of 50% acetaminophen, 23% microcrystalline cellulose (MCC), 22% dextrose and 5% starch (by weight of intragranular portion).
***Including a granulation of 1% dextromethorphan, 48% microcrystalline cellulose (MCC), 46% dextrose and 5% starch (by weight of intragranular portion).
****Including a granulation of 1% phenylephrine HCl, 1% dextromethorphan, 48% acetaminophen, 23% microcrystalline cellulose (MCC), 22% dextrose and 5% starch (by weight of intragranular portion).

### Process Flow

The compositions indicated in the above Tables 1A and 1B and 2 are each processed into two-layer tablets with compositions as outlined in examples 1-12, 13-24 and 25-36. In other words, the examples 1-12 are bi-layer tablets with a first layer according to Table 1A and 1B and the second layer is based on the components listed in Table 2, column 1 (i.e. primarily DC Maltitol). In examples 13-24, the second layer is based on the components listed in Table 2, column 2 (i.e. primarily DC Xylitol). In examples 25-36, the second layer is based on the components listed in Table 2, column 3 (i.e. primarily based on the intragranular portion).

The composition of Table 3 is likewise processed into corresponding two-layer tablets of each of the compositions as indicated examples 37, 37A, 38, 38A, 38B and 38C.

For each example 1-38, 37A, 38A, 38B and 38C the raw materials are sieved with a 1600 micron sieve and then weighed into the proper amount according to the exampled compositions of Tables 1A to 3.

The weighed amounts are then added to a Turbula mixer in a stainless-steel container and blended at 50 rpm for 5 minutes. Magnesium Stearate (MgSt) was added after 4 minutes of blending. A glidant such as silicon dioxide may optionally be added at this step too.

The mixtures are then tableted by means of a Piccola RIVA DC-SC-041-2 or a Fette 3090i.

The applied molds have circular cross sections with diameters of 16 mm and are hollowed to produce tablets, which are concave and/or curved. Evidently, other mold size and shapes may be applied within the scope of the invention.

The resulting tablets according to Examples 1-38, 37A, 38A, 38B and 38C are then obtained by tableting with a suitable pressure force.

For each tablet of examples 1-38, 37A, 38A, 38B and 38C, the second layer as outlined in Table 2 and referred to as the second layer in Table 3 is pressed initially at a first relatively low pressure of approx. 3 kN. The blended composition of the so-called first layer, i.e. compositions of Tables 1A and 1B and the first layer of Table 3 is then fed to the mold and a final two-layer tablet is then compressed at higher pressure (approx. 20 kN) than the pressure applied on the first layers, thereby producing final two-layer tablets according to Examples 1-38, 37A, 38A, 38B and 38C. It is noted that the final two-layer tablets of examples 1-38, 37A, 38A, 38B and 38C are 1.8 grams tablets and that the first layer of the tablets weighs 0.9 and the second layer of the tablets weighs 0.9 gram.

A specification of relevant compounds applied in the examples explained above are listed below.

HPC: Hydroxy propyl cellulose. Klucel Nutra D from Ashland
Non-DC Xylitol: Xivia C from Dupont
Non-granulated Sorbitol: PharmSorbidex from Cargill
Non-DC Isomalt: Isomalt GS from Beneo Paltinit
Non-DC Mannitol: C*PharmMannidex 16700 from Cargill
Non-DC Maltitol: Maltisorb P200 from Roquette
Non-DC Erythritol: Zerose 16952 from Cargill
Non-DC Lactitol: Lactitol from Dupont
DC Erythritol: Zerose 16966 from Cargill
DC Xylitol: Xylitab 200 from Dupont
DC Isomalt: Isomalt DC 101 from Beneo Paltinit
DC Mannitol: Pearlitol 200SD from Roquette
DC Maltitol: Sweetpearl 300 DC from Roquette
Non-DC fructose: Fructose M from ADM
Non-DC dextrose: C*PharmDex02011 from Cargill
Non-DC saccharose: Saccharose from Nordzucker
Microcrystalline cellulose (MCC): Avicel PH101 from FMC
Lactose: Pharmatose 200M from DFE Pharma
Starch: Starch 1500® from Colorcon
Magnesium stearate: MF-2-V from Peter Greven
Silicon dioxide: Aerosil 200 from Evonik
Phenylephrine HCl: Batch no. P70-19001 from Syn-Tech
Dextromethorphan: Batch no. EVS11884 from Wockhardt
Acetaminophen: Batch no. 180830C1 from Novacyl

The procedure of granulation of the intragranular portion was as follows:

The various ingredients for use in the intragranular portion were weighted in specified amounts and sieved through a 1 mm sieve before being added to a high shear mixer available from Diosna. This mixture was mixed at a speed of about 250 rpm (impeller speed) and about 300 rpm (chopper speed) for about 3 minutes. The loss on drying (LOD) of the blend was about 3% after this step.

Thereafter, the mixture was wetted and granulated with purified water being slowly added to give a wetting percentage of about 20-30%. Here, the blend was processed with a speed of about 300 rpm (impeller speed) and about 500 rpm (chopper speed) for about 15 minutes. The wet granules were screened through a 2 mm screen using an oscillating calibrator available from ERWEKA.

Then the screened wet granules were dried using a fluidized bed device available from Glatt. The inlet temperature was about 50 Degree Celsius with an air flow rate of above 40 m3/h, a product temperature of about 28 Degree Celsius and an outlet temperature of about 28 Degree Celsius. The dried granular particles had a loss on drying (LOD) of approx. 3%. Subsequently, the dried granules were sieved through a 1 mm sieve and stored.

### Examples 39-41

TABLE 3B
Compositions for 1.8 gram oral tablets.
Amounts are given in wt-% of the tablet.

| Rw material | | | | |
|---|---|---|---|---|
| (wt %) l layer | Ex39 | Ex40 | Ex41 | Ex41A |
| Intragranular portion 1* | 45 | — | — | — |
| Intragranular portion 2* | — | 45 | — | — |
| Intragranular portion 3* | — | — | 45 | — |
| Intragranular portion 4* | — | — | — | 45 |
| Non-DC Erythritol | 48.75 | 48.75 | 48.75 | 48.75 |
| Flavor | 4 | 4 | 4 | 4 |
| HIS | 0.25 | 0.25 | 0.25 | 0.25 |
| Magnesium Stearate | 1 | 1 | 1 | 1 |
| Binder HPC | 1 | 1 | 1 | 1 |

*Including a granulation of 1% phenylephrine HCl, 48% microcrystalline cellulose (MCC), 46% dextrose and 5% starch (by weight of intragranular portion).
**Including a granulation of 50% acetaminophen, 23% microcrystalline cellulose (MCC), 22% dextrose and 5% starch (by weight of intragranular portion).
***Including a granulation of 1% dextromethorphan, 48% microcrystalline cellulose (MCC), 46% dextrose and 5% starch (by weight of intragranular portion).
****Including a granulation of 1% phenylephrine HCl, 1% dextromethorphan, 48% acetaminophen, 23% microcrystalline cellulose (MCC), 22% dextrose and 5% starch (by weight of intragranular portion).

All ingredients were received in powder form.

DC Isomalt-Isomalt DC 101 from Beneo Paltinit
Non-DC Erythritol: Zerose 16952 from Cargill
HPC: Hydroxy propyl cellulose. Klucel Nutra D from Ashland
Microcrystalline cellulose (MCC): Avicel PH101 from FMC
Lactose: Pharmatose 200M from DFE Pharma
Starch: Starch 1500® from Colorcon
Magnesium stearate: MF-2-V from Peter Greven
Silicon dioxide: Aerosil 200 from Evonik
Phenylephrine HCl: Batch no. P70-19001 from Syn-Tech
Dextromethorphan: Batch no. EVS11884 from Wockhardt
Acetaminophen: Batch no. 180830C1 from Novacyl

The procedure of granulation of the intragranular portion was as follows:

The various ingredients for use in the intragranular portion were weighted in specified amounts and sieved through a 1 mm sieve before being added to a high shear mixer available from Diosna. This mixture was mixed at a speed of about 250 rpm (impeller speed) and about 300 rpm (chopper speed) for about 3 minutes. The loss on drying (LOD) of the blend was about 3% after this step.

Thereafter, the mixture was wetted and granulated with purified water being slowly added to give a wetting percentage of about 20-30%. Here, the blend was processed with a speed of about 300 rpm (impeller speed) and about 500 rpm (chopper speed) for about 15 minutes. The wet granules were screened through a 2 mm screen using an oscillating calibrator available from ERWEKA.

Then the screened wet granules were dried using a fluidized bed device available from Glatt. The inlet temperature was about 50 Degree Celsius with an air flow rate of above 40 m3/h, a product temperature of about 28 Degree Celsius and an outlet temperature of about 28 Degree Celsius. The dried granular particles had a loss on drying (LOD) of approx. 3%. Subsequently, the dried granules were sieved through a 1 mm sieve and stored.

### Process Flow

For each of the examples 39-41 and 41A the raw materials are sieved with a 1600 micron sieve and then weighed into the proper amount according to the exampled compositions of Table 3B.

For each example the weighed amounts are then added to a Turbula mixer in a stainless-steel container and blended at 50 rpm for 4 minutes and then adding magnesium stearate and blending one additional minute. A glidant such as silicon dioxide may optionally be added at this step too.

The resulting tablets according to Examples 39-41 and 41A are then obtained by tableting the mixtures by means of a Piccola RIVA DC-SC-041-2. A Fette 3090i may also applied.

### Evaluation

TABLE 3CA
Sensory evaluation of Examples 13-18.

| | Total sensory | | Initial |
|---|---|---|---|
| | experience | | Watering |
| | Very good/Good/ | | effect 1-5 |
| | Acceptable(Acc)/ | | 1 low |
| Ex | Poor | Evaluation | 5 high |
| 13 | Good | Good initial chew, not too hard, | 5 |
| | | disintegrates with crunchy feeling, | |
| | | many big particles for a long time | |
| 14 | Poor | Unacceptable hard chew - not | 1 |
| | | chewable and poorly dissolvable | |
| | | within the first 30 seconds. | |
| 15 | Good | Slightly hard initial chew, but OK | 4 |
| | | and acceptable disintegration. Saliva | |
| | | increases suitably but with many big | |
| | | non-dissolved particles though the | |
| | | first 30 sec. | |
| 16 | Very Good | Excellent crunchy fast dissolving | 5 |
| | | tablet | |
| 17 | Acceptable | Soft initial chew, different mouth | 3 |
| | | feel. Dissolves adequately fast and | |
| | | provide OK watering effect | |
| 18 | Good | Slightly hard initial chew, but OK | 5 |
| | | and acceptable disintegration. | |
| | | Salivation generation good but a little | |
| | | sandy liquid feeling | |

TABLE 3CB
Sensory evaluation of Examples 19-24.

| | Total sensory | | Initial |
|---|---|---|---|
| | experience | | Watering |
| | Very good/Good/ | | effect 1-5 |
| | Acceptable(Acc)/ | | 1 low |
| Ex | Poor | Evaluation | 5 high |
| 19 | Good | OK and suitable disintegration. Saliva | 4 |
| | | increases suitably but with some non- | |
| | | dissolved particles though the first | |
| | | 30 sec. | |
| 20 | Acceptable | Acceptable hard initial chew, OK | 3 |
| | | watering effect. | |
| 21 | Acceptable | OK initial chew. Dissolves | 3 |
| | | adequately fast and provide OK | |
| | | watering effect | |
| 22 | Very Good | Excellent crunchy, fast dissolving and | 5 |
| | | watery tablet | |
| 23 | Good | Fine and suitable disintegration. | 4 |
| | | Saliva increases suitably but with | |
| | | many non-dissolved particles though | |
| | | the first 30 sec. | |
| 24 | Acceptable | Salivation generation OK but less | 3 |
| | | optimal compared to tablets with an | |
| | | intragranular portion | |

TABLE 3CC
Sensory evaluation of Examples 25-30.

| | Total sensory | | Initial |
|---|---|---|---|
| | experience | | Watering |
| | Very | | effect |
| | good/Good/ | | 1-5 |
| | Acceptable | | 1 low |
| Ex | (Acc)/Poor | Evaluation | 5 high |
| 25 | Very Good | Fine initial chew, disintegrate with | 5 |
| | | pleasant feeling, a number of big | |
| | | particles for a long time | |
| 26 | Poor | Unacceptable hard chew - not chewable | 1 |
| | | and poorly dissolvable within the first | |
| | | 30 seconds. | |
| 27 | Very good | Fine and suitable disintegration. Saliva | 4 |
| | | increases suitably but with some big | |
| | | non-dissolved particles though the | |
| | | first 30 sec. | |
| 28 | Very Good | Excellent crunchy fast dissolving tablet | 5 |
| 29 | Acceptable | Soft initial chew, different mouth feel. | 3 |
| | | Dissolves adequately fast and provide | |
| | | OK watering effect | |
| 30 | Very good | Salivation generation excellent but a | 5 |
| | | little sandy liquid feeling | |

TABLE 3CD
Sensory evaluation of Examples 31-36.

| | Total sensory | | Initial |
|---|---|---|---|
| | experience | | Watering |
| | Very | | effect |
| | good/Good/ | | 1-5 |
| | Acceptable | | 1 low |
| Ex | (Acc)/Poor | Evaluation | 5 high |
| 31 | Very good | Fine and suitable disintegration. Saliva | 4 |
| | | increases nicely | |
| 32 | Acceptable | Slightly hard initial chew, OK watering | 3 |
| | | effect. | |
| 33 | Very good | Nice and good disintegration. Saliva | 4 |
| | | increases but with a number of big | |
| | | non-dissolved particles though the | |
| | | first 30 sec. | |
| 34 | Very Good | Excellent crunchy, fast dissolving tablet | 5 |
| | | and watery tablet | |
| 35 | Good | Fine and suitable disintegration. Saliva | 4 |
| | | increases suitably but with many big | |
| | | non-dissolved particles though the | |
| | | first 30 sec. | |
| 36 | Very good | Salivation generation surprisingly good | 5 |
| | | and very good texture of the tablet | |

TABLE 3CE
Sensory evaluation of Examples 37-38, 37A, 38A, 38B and 38C.

| | Total sensory | | Initial |
|---|---|---|---|
| | experience | | Watering |
| | Very | | effect |
| | good/Good/ | | 1-5 |
| | Acceptable | | 1 low |
| Ex | (Acc)/Poor | Evaluation | 5 high |
| 37 | Very good | Salivation generation very good and very | 5 |
| | | good texture of the tablet | |
| 37A | Very Good | Good initial chew, fast dissolving tablet | 4 |
| | | and watery tablet | |
| 38 | Very Good | Excellent crunchy, fast dissolving tablet | 4 |
| | | and watery tablet | |
| 38A | Very good | Salivation generation surprisingly good | 5 |
| | | and surprisingly good texture of the | |
| | | tablet | |
| 38B | Very Good | Excellent crunchy, fast dissolving tablet | 4 |
| | | and watery tablet | |
| 38C | Good | Good initial chew, fast dissolving tablet | 4 |
| | | and watery tablet | |

TABLE 3CF
Sensory evaluation of Examples 39-41 and 41A.

| | Total sensory | | Initial |
|---|---|---|---|
| | experience | | Watering |
| | Very | | effect |
| | good/Good/ | | 1-5 |
| | Acceptable | | 1 low |
| Ex | (Acc)/Poor | Evaluation | 5 high |
| 39 | Very good | Salivation generation surprisingly good | 5 |
| | | and surprisingly good texture of the | |
| | | tablet | |
| 40 | Very Good | Excellent crunchy, fast dissolving tablet | 4 |
| | | and watery tablet | |
| 41 | Very good | Salivation generation very good and | 5 |
| | | very good texture of the tablet | |
| 41A | Good | Good initial chew, sufficiently fast | 4 |
| | | dissolving tablet and good watery effect | |

The above two-layer tablets as well as one-layered tablets were evaluated according to three parameters by a test panel.

Two of the parameters (texture and disintegration) were suitability as a chewable tablet and one parameter was the perceived watering effect. Due to the more complex nature of a two-layer tablet two further parameters were evaluated, namely resistance to crunch and friability. However, these results are not shown.

It was first of all noted that the watering effect was considered relatively high for the inventive examples. The watering effect is considered to be representative or equal to the elsewhere described salivation effect (or generation of saliva).

The test panel clearly indicated that the overall chewing process and the mouthfeel was no less than impressive in relation to various of the inventive examples. It was also noted that the test panel identified inventive examples to provide an impressive watering effect when compared to e.g. the sorbitol-based example.

With respect to two-layered tablets with an intragranular portion in layer 1, impressive results were seen compared for tablets where the DC sweeteners were replaced completely or in part with the intragranular portion.

Additionally, with respect to two-layered tablets, when the intragranular portion was present in layer 2 very impressive results were also seen compared to tablets where layer 2 was mainly composed of DC sweeteners.

In the situation where the intragranular portion was only contained in layer 2 and not in layer 1, surprising results were seen—in some case even better than when the intragranular portion was also present in layer 1.

With respect to one-layered tablets, the results were surprisingly impressive, both in terms of watering effect and tablet characteristics.

### Examples 42-44 Preparation with Different Levels of Non-DC Sugar Alcohol

TABLE 5
Oral tablet compositions for bi-layered tablets.

| | Ex42 | Ex43 | Ex44 |
|---|---|---|---|
| Raw material | | | |
| (wt %) layer 1 | | | |
| Non-DC | 5 | 50 | 82 |
| Erythritol | | | |
| Intragranular | 90.8 | 43.8 | 9.8 |
| portion* | | | |
| Flavor | 3 | 3 | 3 |
| HIS | 0.2 | 0.2 | 0.2 |
| Binder HPC | 1 | 3 | 5 |
| Raw material | | | |
| (wt %) layer 2 | | | |
| DC Xylitol | 96.9 | 96.9 | 96.9 |
| Flavor | 3 | 3 | 3 |
| HIS | 0.1 | 0.1 | 0.1 |

The ratio of layer 1 to layer 2 is 55:45. The tablet weight is 1.5 g. Hence the weight of layer 1 is 0.825 g whereas the weight of layer 2 is 0.675 g. Amounts are given in wt-% of the respective layer of the tablet *Including a granulation of 2% dextromethorphan, 47% microcrystalline cellulose (MCC), 46% dextrose and 5% starch (by weight of intragranular portion).

### Examples 45-48 Preparation with Different Levels of Non-DC Sugar Alcohol

TABLE 6
Oral tablet compositions for bi-layered tablets.

| | Ex45 | Ex46 | Ex47 | Ex48 |
|---|---|---|---|---|
| Raw material | | | | |
| (wt %) layer 1 | | | | |
| DC Erythritol | 0 | 20 | 30 | 40 |
| Non-DC Erythritol | 50 | 30 | 20 | 10 |
| DC Isomalt | 43.8 | 43.8 | 43.8 | 43.8 |
| Flavor | 3 | 3 | 3 | 3 |
| HIS | 0.2 | 0.2 | 0.2 | 0.2 |
| Binder HPC | 3 | 3 | 3 | 3 |
| Raw material | | | | |
| (wt %) layer 2 | | | | |
| Intragranular | 96.9 | 96.9 | 96.9 | 96.9 |
| portion* | | | | |
| Flavor | 3 | 3 | 3 | 3 |
| HIS | 0.1 | 0.1 | 0.1 | 0.1 |

The ratio of layer 1 to layer 2 is 55:45. The tablet weight is 1.5 g. Hence the weight of layer 1 is 0.825 g whereas the weight of layer 2 is 0.675 g. Amounts are given in wt-% of the respective layer of the tablet. *Including a granulation of 1% phenylephrine HCl, 48% microcrystalline cellulose (MCC), 46% dextrose and 5% starch.

### Process Flow

The compositions indicated in the above Tables 5 and 6 are each processed into two-layer tablets with compositions as outlined in examples 42-48.

For each example 42-48 the raw materials are sieved with a 1600 micron sieve and then weighed into the proper amount according to the exampled compositions.

The weighed amounts are then added to a Turbula mixer in a stainless-steel container and blended at 50 rpm for 5 minutes. Magnesium Stearate (MgSt) was added after 4 minutes of blending. A glidant such as silicon dioxide may optionally be added at this step too.

The mixtures are then tableted by means of a Piccola RIVA DC-SC-041-2 or a Fette 3090i.

The applied molds have circular cross sections with diameters of 16 mm and are hollowed to produce tablets, which are concave and/or curved. Evidently, other mold size and shapes may be applied within the scope of the invention.

The resulting tablets according to Examples 42-48 are then obtained by tableting with a suitable pressure force.

For each tablet of examples 42-48, the second layer is pressed initially at a first relatively low pressure of approx. 3 kN. The blended composition of the so-called first layer is then fed to the mold and a final two-layer tablet is then compressed at higher pressure (approx. 20 kN) than the pressure applied on the first layers, thereby producing final two-layer tablets.

It is noted that the final two-layer tablets of examples 42-48 are 1.5 grams tablets and the ratio of layer 1 to layer 2 is 55:45. The tablet weight is 1.5 g. Hence the weight of layer 1 is 0.825 g whereas the weight of layer 2 is 0.675 g.

A specification of relevant compounds applied in the examples explained above are listed below.

HPC: Hydroxy propyl cellulose. Klucel Nutra D from Ashland
Non-DC Isomalt: Isomalt GS from Beneo Paltinit
Non-DC Erythritol: Zerose 16952 from Cargill
DC Erythritol-Zerose 16966 from Cargill
DC Xylitol-Xylitab 200 from Dupont
DC Isomalt-Isomalt DC 101 from Beneo Paltinit
Microcrystalline cellulose (MCC): Avicel PH101 from FMC
Lactose: Pharmatose 200M from DFE Pharma
Starch: Starch 1500® from Colorcon
Magnesium stearate: MF-2-V from Peter Greven
Silicon dioxide: Aerosil 200 from Evonik
Phenylephrine HCl: Batch no. P70-19001 from Syn-Tech
Dextromethorphan: Batch no. EVS11884 from Wockhardt

The procedure of granulation of the intragranular portion was as follows:

The various ingredients for use in the intragranular portion were weighted in specified amounts and sieved through a 1 mm sieve before being added to a high shear mixer available from Diosna. This mixture was mixed at a speed of about 250 rpm (impeller speed) and about 300 rpm (chopper speed) for about 3 minutes. The loss on drying (LOD) of the blend was about 3% after this step.

Thereafter, the mixture was wetted and granulated with purified water being slowly added to give a wetting percentage of about 20-30%. Here, the blend was processed with a speed of about 300 rpm (impeller speed) and about 500 rpm (chopper speed) for about 15 minutes. The wet granules were screened through a 2 mm screen using an oscillating calibrator available from ERWEKA.

Then the screened wet granules were dried using a fluidized bed device available from Glatt. The inlet temperature was about 50 Degree Celsius with an air flow rate of above 40 m3/h, a product temperature of about 28 Degree Celsius and an outlet temperature of about 28 Degree Celsius. The dried granular particles had a loss on drying (LOD) of approx. 3%. Subsequently, the dried granules were sieved through a 1 mm sieve and stored.

### Test Set-Up

The above two-layer Examples 42-48 were evaluated with respect to watering effect by a test panel in a time-intensity evaluation set-up. The watering effect is the perceived watering effect by the test panel, i.e. the overall impression of watering effect upon oral administration and commencement of the test.

The test set-up was composed of 8 test persons in a test panel with 2 repetitions of each variant. Each of the test persons were healthy individuals appointed on an objective basis according to specified requirements. The sensory analysis was performed according to ISO 4121-2003 in testing conditions following ISO 8589. The result is an average of the results of the 8 individuals for 2 repetitions, giving a total of 16 measurements for each variant.

The 8 test individuals were instructed to swallow saliva before the test was commenced and was not allowed to swallow during testing. The test individuals chewed the samples with a frequency of about 60 chews pr. minute. After 30 seconds, the saliva generated was collected and the weight of the saliva was noted. Before a new test was conducted, the oral cavity was rinsed with water and a time gap of 4 minutes before next test was complied with. Each sample was tested twice by each individual in the test. Hence a total of 16 test results were generated for each sample. Each test individual was allowed to test 6 samples in a test series. At least 30 minutes gap between each test series was complied with.

The test individuals indicated watering effect intensity on a scale from 0 to 10 where 10 indicates the highest watering effect. The watering effect is measured as a function of time in seconds.

Due to the more complex nature of a two-layer tablet two further parameters were evaluated, namely resistance to crunch and friability. The resistance to crunch and friability was measured and indicated in the Examples 42-48.

The resistance to crunch is determined according to European Pharmacopoeia 9.1, test method 2.9.8. by using a pharmaceutical resistance to crunch tester model Pharma Test type PTB 311.

Friability is measured according to European Pharmacopoeia 9.1, test method 2.9.7. by using a pharmaceutical friability-tester PTF 10E from Pharma Test.

### Watering Effect

TABLE 7
Results of watering effect based on Examples 42-44.

| Watering effect | Ex42 | Ex43 | Ex44 |
|---|---|---|---|
| 15 s | 7.0 | 8.0 | 8.5 |
| 20 s | 8.0 | 8.5 | 9.0 |
| 25 s | 9.0 | 9.5 | 10.0 |

The values indicate watering effect intensity on a scale from 0 to 10 where 10 is the highest score on watering effect. The watering effect is measured as a function of time in seconds. Samples were chewed for 30 seconds without swallowing.

The results indicate that a low amount of the intragranular portion (Ex44) gives a more pronounced watering effect and a high amount the intragranular portion (Ex42) give a slightly lower watering effect. This was expected since the non-DC sweeteners were expected to serve the purpose of watering. However, it was not expected that even a very high amount of the intragranular portion gave an acceptable watering effect.

TABLE 8
Results of watering effect based on Examples 45-48.

| Watering effect | Ex45 | Ex46 | Ex47 | Ex48 |
|---|---|---|---|---|
| 10 s | 8.5 | 7.0 | 5.0 | 2.5 |
| 20 s | 9.0 | 8.0 | 6.5 | 4.0 |
| 25 s | 10.0 | 9.0 | 7.0 | 6.0 |

The values indicate watering effect intensity on a scale from 0 to 10 where 10 is the highest score on watering effect. The watering effect is measured as a function of time in seconds. Samples chewed for 30 seconds without swallowing.

The results indicate that a high amount of the intragranular portion in layer 2 is very beneficial.

### Examples 49-63. Preparation of Intragranular Components with Actives

TABLE 9
Amounts are given in wt-% of the respective layer of composition.

| Raw material | | | | | |
|---|---|---|---|---|---|
| (wt %) | Ex49 | Ex50 | Ex51 | Ex52 | Ex53* |
| Phenylephrine HCl | 1 | 1 | 1 | 1 | 1 |
| Microcrystalline | 48 | 48 | 48 | 48 | 48 |
| cellulose (MCC) | | | | | |
| Non-DC Lactose | 46 | — | — | — | — |
| Starch | 5 | 5 | 5 | 5 | 5 |
| Non-DC Dextrose | — | 46 | — | — | — |
| Non-DC Erythritol | — | — | 46 | — | — |
| Non-DC Mannitol | — | — | — | 46 | — |
| DC Mannitol | — | — | — | — | 46 |

*This composition is a comparative composition where the individual components were not granulated and later applied directly in direct compression.

TABLE 10
Amounts are given in wt-% of the respective layer of composition.

| Raw material | | | | | |
|---|---|---|---|---|---|
| (wt %) | Ex54 | Ex55 | Ex56 | Ex57 | Ex58* |
| Acetaminophen | 50 | 50 | 50 | 50 | 50 |
| Microcrystalline | 23 | 23 | 23 | 23 | 23 |
| cellulose (MCC) | | | | | |
| Non-DC Lactose | 22 | — | — | — | — |
| Starch | 5 | 5 | 5 | 5 | 5 |
| Non-DC Dextrose | — | 22 | — | — | — |
| Non-DC Erythritol | — | — | 22 | — | — |
| Non-DC Mannitol | — | — | — | 22 | — |
| DC Mannitol | — | — | — | — | 22 |

*This composition is a comparative composition where the individual components were not granulated and later applied directly in direct compression.

TABLE 11
Amounts are given in wt-% of the respective layer of composition.

| Raw material | | | | | |
|---|---|---|---|---|---|
| (wt %) | Ex59 | Ex60 | Ex61 | Ex62 | Ex63* |
| Dextromethorphan | 1 | 1 | 1 | 1 | 1 |
| Microcrystalline | 48 | 48 | 48 | 48 | 48 |
| cellulose (MCC) | | | | | |
| Non-DC Lactose | 46 | — | — | — | — |
| Starch | 5 | 5 | 5 | 5 | 5 |
| Non-DC Dextrose | — | 46 | — | — | — |
| Non-DC Erythritol | — | — | 46 | — | — |
| Non-DC Mannitol | — | — | — | 46 | — |
| DC Mannitol | — | — | — | — | 46 |

*This composition is a comparative composition where the individual components were not granulated and later applied directly in direct compression.

TABLE 12
Amounts are given in wt-% of the respective layer of composition.

| Raw material | | | | | |
|---|---|---|---|---|---|
| (wt %) | Ex64 | Ex65 | Ex66 | Ex67 | Ex68* |
| Phenylephrine | 1 | 1 | 1 | 1 | 1 |
| Dextromethorphan | 1 | 1 | 1 | 1 | 1 |
| Acetaminophen | 48 | 48 | 48 | 48 | 48 |
| Microcrystalline | 23 | 23 | 23 | 23 | 23 |
| cellulose (MCC) | | | | | |
| Non-DC Lactose | 22 | — | — | — | — |
| Starch | 5 | 5 | 5 | 5 | 5 |
| Non-DC Dextrose | — | 22 | — | — | — |
| Non-DC Erythritol | — | — | 22 | — | — |
| Non-DC Mannitol | — | — | — | — | — |
| DC Mannitol | — | — | — | — | 22 |

*This composition is a comparative composition where the individual components were not granulated and later applied directly in direct compression.

Non-DC Lactose: Pharmatose 200M from DFE Pharma
Non-DC Dextrose: C*PharmDex02011 from Cargill
Non-DC Erythritol: C*Pharm Erythritol 16956 from Cargill
Non-DC Mannitol: C*PharmMannidex 16700 from Cargill
DC Mannitol: Pearlitol 300 DC from Roquette
Microcrystalline cellulose (MCC): Avicel PH101 from FMC
Starch: Starch 1500® from Colorcon
Magnesium stearate: MF-2-V from Peter Greven
Silicon dioxide: Aerosil 200 from Evonik
Phenylephrine HCl: Batch no. P70-19001 from Syn-Tech
Dextromethorphan: Batch no. EVS11884 from Wockhardt
Acetaminophen: Batch no. 180830C1 from Novacyl

The procedure of granulation of the intragranular portion was as follows:

The various ingredients for use in the intragranular portion were weighted in specified amounts and sieved through a 1 mm sieve before being added to a high shear mixer available from Diosna. This mixture was mixed at a speed of about 250 rpm (impeller speed) and about 300 rpm (chopper speed) for about 3 minutes. The loss on drying (LOD) of the blend was about 3% after this step.

Thereafter, the mixture was wetted and granulated with purified water being slowly added to give a wetting percentage of about 20-30%. Here, the blend was processed with a speed of about 300 rpm (impeller speed) and about 500 rpm (chopper speed) for about 15 minutes. The wet granules were screened through a 2 mm screen using an oscillating calibrator available from ERWEKA.

Then the screened wet granules were dried using a fluidized bed device available from Glatt. The inlet temperature was about 50 Degree Celsius with an air flow rate of above 40 m3/h, a product temperature of about 28 Degree Celsius and an outlet temperature of about 28 Degree Celsius. The dried granular particles had a loss on drying (LOD) of approx. 3%. Subsequently, the dried granules were sieved through a 1 mm sieve and stored.

The intragranular portions indicated in the above Table 9-12 were analyzed with respect to uniformity of content of the drug substances.

The method used for content uniformity of in the intragranular portion samples is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

Specifically, a series of at least 5 samples having a weight of 1.0 g were measured with respect to content of uniformity.

Each of these intragranular portion samples were thereafter processed into tablets by including Aerosil 200 in a small amount and magnesium stearate (MgSt) also in a small amount (less than 1% by weight of the compositions above for both). The tablets were made into one-layered tablets.

For each tablet sample, the raw materials are sieved with a 1600 micron sieve and then weighed into the proper amount. The weighed amounts are then added to a Turbula mixer in a stainless-steel container and blended at 50 rpm for 5 minutes. MgSt was added after 4 minutes of blending. The mixtures are then tableted by means of a Piccola RIVA DC-SC-041-2 or a Fette 3090i. The applied molds have circular cross sections with diameters of 13 mm and are hollowed to produce tablets, which are concave and/or curved. Evidently, other mold size and shapes may be applied within the scope of the invention.

The tablets were also analyzed with respect to uniformity of content.

The method used for content uniformity of tablets is determined according to European Pharmacopoeia 10.8 when using test method 2.9.40. Uniformity of dosage units. The acceptance value (AV) is calculated using mass variation (MV) or content uniformity (CU) depending on the dose and ratio of the drug substance. An appropriate analytical method is selected for content uniformity.

Specifically, a series of at least 10 tablets having a weight of about 700 mg were measured with respect to uniformity of content.

With respect to the tablet samples, measurements of uniformity of content were made on samples collected at specific time points during the tableting process (stratified sampling), i.e. samples for analysis were collected after 10, 20 and 30 minutes of tableting.

These measurements were made for all samples in Tables 9-12 and all tablets based on the samples in Tables 9-12. Measurements for the intragranular portions of Table 9 is shown below:

TABLE 13
Content of uniformity measurements for intragranular samples.

| Uniformity of content | Ex49 | Ex50 | Ex51 | Ex53* |
|---|---|---|---|---|
| Average API content | 95 | 93 | 89 | 95 |
| RSD (%) | 1 | 1 | 1 | 1 |
| Min (%) | 92 | 92 | 87 | 94 |
| Max (%) | 96 | 94 | 89 | 96 |

*This composition is a comparative composition where the individual components were not granulated and later applied directly in direct compression.

TABLE 14
Content of uniformity measurements for tablets (pooled samples)
based on the intragranular samples.

| Uniformity of content | Ex49 | Ex50 | Ex51 | Ex53* |
|---|---|---|---|---|
| Average API content | 97 | 97 | 95 | 88 |
| RSD (%) | 8 | 2 | 1 | 8 |
| Min (%) | 77 | 93 | 93 | 77 |
| Max (%) | 98 | 99 | 97 | 98 |

*This composition is a comparative composition where the individual components were not granulated and later applied directly in direct compression.

TABLE 15
Content of uniformity measurements for tablets (stratified sampling)
based on the intragranular samples.

| Uniformity of content | Ex49 | Ex50 | Ex51 | Ex53* |
|---|---|---|---|---|
| After 10 minutes | 98 | 98 | 100 | 91 |
| After 20 minutes | 97 | 97 | 95 | 84 |
| After 30 minutes | 96 | 99 | 95 | 73 |
| Difference after 30 min | 2 | 1 | 5 | 18 |

*This composition is a comparative composition where the individual components were not granulated and later applied directly in direct compression.

What is claimed is:

1. An oral tablet suitable for active pharmaceutical ingredients comprising an intragranular portion and an extragranular portion,
the intragranular portion comprising a plurality of granules each comprising:
one or more active pharmaceutical ingredients;
one or more binders; and
one or more excipients comprising one or more sweeteners in an amount of at least 20% by weight of the plurality of granules,
the extragranular portion comprising a population of particles comprising:
non-directly compressible (non-DC) sweetener particles, the non-DC sweetener particles having not been granulated prior to tableting and providing the oral tablet with a plurality of discrete non-DC areas evenly distributed in the oral tablet or at least one module of the oral tablet.

2. The oral tablet according to claim 1, wherein the oral tablet is a chewable tablet.

3. The oral tablet according to claim 1, wherein the extragranular portion constitutes at least 20% by weight of the oral tablet.

4. The oral tablet according to claim 1, wherein the intragranular portion constitutes at least 30% by weight of the oral tablet.

5. The oral tablet according to claim 1, wherein saliva generation upon mastication of the oral tablet is increased compared to an oral tablet where the discrete areas of the extragranular portion are based on DC sweetener particles.

6. The oral tablet according to claim 1, wherein the non-DC sweetener particles of the extragranular portion is present in an amount of at least 10% by weight of the oral tablet.

7. The oral tablet according to claim 1, wherein the non-DC sweetener particles of the extragranular portion are selected from the group consisting of non-DC particles of erythritol, maltitol, xylitol, isomalt, lactitol, mannitol, and combinations thereof.

8. The oral tablet according to claim 1, wherein the extragranular portion further comprises DC sweetener particles, including one or more DC sugar alcohol particles or one or more DC saccharide particles.

9. The oral tablet according to claim 1, wherein the one or more active pharmaceutical ingredients comprises one or more low dose active pharmaceutical ingredients in an amount of less than 4% by weight of the oral tablet.

10. The oral tablet according to claim 1, wherein the one or more active pharmaceutical ingredients comprises one or more low dose active pharmaceutical ingredients selected from the group consisting of diphenhydramine, cetirizine, loratadine, chlorpheniramine maleate, levocetirizine, meclizine, dextromethorphan, phenylephrine, famotidine, omeprazole, doxylamine succinate, melatonin, and any combination and mixture thereof.

11. The oral tablet according to claim 1, wherein the one or more binders of the intragranular portion is present in an amount of 0.1 to 15% by weight of the intragranular portion.

12. The oral tablet according to claim 1, wherein the one or more binders of the intragranular portion is selected from the group consisting of hydroxypropyl cellulose (HPC), hydroxypropylmethyl cellulose (HPMC), carboxymethyl cellulose (CMC), low substituted hydroxypropyl cellulose (L-HPC), polyvinylpyrrolidone (PVP), copovidone, and any mixture and combination thereof.

13. The oral tablet according to claim 1, wherein the one or more excipients comprising one or more sweeteners of the intragranular portion is selected from the group consisting of erythritol, maltitol, xylitol, isomalt, lactitol, mannitol, sorbitol and combinations thereof.

14. The oral tablet according to claim 1, wherein the one or more active pharmaceutical ingredients comprises one or more medium dose active pharmaceutical ingredients in an amount of 4 to 30% by weight of the oral tablet.

15. The oral tablet according to claim 1, wherein the one or more active pharmaceutical ingredients comprises one or more medium dose active pharmaceutical ingredients selected from the group consisting of diphenhydramine, fexofenadine, dimenhydrinate, meclizine, pseudoephedrine, aspirin, caffeine, theanine, and any combination and mixture thereof.

16. The oral tablet according to claim 1, wherein the one or more active pharmaceutical ingredients comprises one or more high dose active pharmaceutical ingredients in an amount of 20 to 60% by weight of the oral tablet.

17. The oral tablet according to claim 1, wherein the one or more active pharmaceutical ingredients comprises one or more high dose active pharmaceutical ingredients selected from the group consisting of fexofenadine, guaifenesin, calcium carbonate, magnesium hydroxide, acetaminophen, aspirin, ibuprofen, naproxen sodium, caffeine, and any combination and mixture thereof.

18. The oral tablet according to claim 1, wherein the intragranular portion further comprises a granulation diluent in an amount of at least 20% by weight of the intragranular portion.

19. The oral tablet according to claim 1, wherein the intragranular portion further comprises a disintegrant.

20. The oral tablet according to claim 1, wherein the oral tablet comprises at least two modules comprising a first tablet module and a second tablet module.

21. The oral tablet according to claim 1, wherein the oral tablet comprises at least two modules comprising a first tablet module and a second tablet module, and wherein the extragranular portion is comprised in the first module and the intragranular portion is comprised in the second module.

22. The oral tablet according to claim 1, wherein a series of at least 10 tablets comprise the one or more active pharmaceutical ingredients in an amount varying with a relative standard deviation (RSD) below 5%.
